(19) United States
(12) Patent Application Publication
(10) Pub. No.: US 2025/0011296 A1
(43) Pub. Date: Jan. 9, 2025

(54) MODULATORS OF MOLECULAR TARGETS EXPRESSED IN METABOLIC AND INFLAMMATORY DISORDERS

(71) Applicants: Gerardo David ANAYA EUGENIO, Columbus, OH (US); Ohio State Innovation Foundation, Columbus, OH (US)

(72) Inventors: Esperanza CARCACHE DE BLANCO, Columbus, OH (US); Gerardo David ANAYA EUGENIO, Columbus, OH (US); Nicole A. WOODARD, Philadelphia, PA (US)

(21) Appl. No.: 18/696,042

(22) PCT Filed: Sep. 29, 2022
(86) PCT No.: PCT/US22/45238

§ 371 Date: Mar. 27, 2024

Related U.S. Application Data
(60) Provisional application No. 63/249,879, filed on Sep. 29, 2021.

(51) Int. Cl. C07D 317/54; A61K 31/343; A61K 31/357; A61K 36/185; A61P 3/10; C07D 307/86; C07D 317/60; C07D 405/04

(52) U.S. Cl. CPC C07D 317/54; A61K 31/343; A61K 31/357; A61K 36/185; A61P 3/10; C07D 307/86; C07D 317/60; C07D 405/04

(57) ABSTRACT

The present disclosure provides compounds that modulate expressed in metabolic and inflammatory disorders targets, including peroxisome proliferator activated receptors (PPARs), phosphoenolpyruvate carboxy kinase (PEPCK), poly(ADP-ribose) polymerases (PARPs), tankyrase (TNKS), nuclear factor kappa-light-chain-enhancer of activated B cells (NF-KB), or glucose transporter type 4 (GLUT-4). Also provided is the use of the disclosed compounds in the treatment of diseases and disorders, for example metabolic disorders and in particular diabetes.

### CROSS-REFERENCE TO RELATED APPLICATIONS

This application claims the benefit of priority to U.S. Provisional Patent Application No. 63/249,879, filed Sep. 29, 2021, the disclosure of which is incorporated herein by reference in its entirety.

### TECHNICAL FIELD

This disclosure relates to compounds for the treatment of metabolic disorders such as diabetes, and more particularly to compounds that modulate metabolic disease targets such as PPAR, PEPCK, PARP-5/TNKS, NF-κB, and Glut-4.

### BACKGROUND

Diabetes has reached alarming endemic levels, affecting over 30 million people in the United States that currently have available only less than optimal treatments. Some known risk factors of the disease are obesity, poor dietary habits, and sedentary lifestyle. The prevalence of risk factors contributing to type 2 diabetes calls for research to develop new safe and effective drugs that do not only respond to the contributing factors, but also treat the disease itself to prevent the disease complications or associated diseases such retinopathy, diabetes nephropathy, and other related diseases. Antidiabetic drugs currently in use have unwanted side effects, such as those observed from thiazolidinediones including weight gain, increased risk of heart attack, and increase in liver enzymes with the potential for triggering inflammatory response.

There is a clear need for new treatment that may be used in the treatment of metabolic disease, in particular diabetes.

### SUMMARY

In accordance with the purposes of the disclosed materials and methods, as embodied and broadly described herein, the disclosed subject matter, in one aspect, relates to compounds and their pharmaceutically acceptable salts, prodrugs, and derivatives thereof, that are useful as modulators of molecular targets expressed in metabolic disorders, for example peroxisome proliferator activated receptors (PPARs), phosphoenolpyruvate carboxykinase (PEPCK), poly(ADP-ribose) polymerases (PARPs), tankyrase (TNKS), nuclear factor kappa-light-chain-enhancer of activated B cells (NF-κB), or glucose transporter type 4 (GLUT-4). Also provided is the use of the disclosed compounds in the treatment of diseases and disorders, for example metabolic disorders and in particular diabetes. Additional advantages will be set forth in the description that follows, and in part will be obvious from the description, or may be learned by practice of the aspects described below. The advantages described below will be realized and attained by means of the elements and combinations particularly pointed out in the appended claims.

The details of one or more embodiments of the disclosure are set forth in the description below. Other features, objects, and advantages of the disclosure will be apparent from the description the claims.

### DESCRIPTION OF DRAWINGS

FIG. 1 provides an extraction scheme for edible plant material.

FIG. 2 provides an M. fragrans extraction scheme of two precipitates (K033 and K034) and F022 for further fractionation.

FIG. 3 depicts the combination of three bilevel fractions (F023, F024, and F044) for reversed-phase column chromatography.

FIG. 4 provides a preliminary Western blot analysis of zebrafish treatment groups: A) healthy, untreated; B) diabetic, untreated; C) diabetic, 10 μM rosiglitazone; D) diabetic, 10 μM mace isolates 41-43.

FIG. 5 provides Western blot band densities for expression levels in zebrafish treatment groups: A) healthy, untreated; B) diabetic, untreated; C) diabetic, 10 μM rosiglitazone; D) diabetic, 10 μM mace isolates 41-43.

FIG. 6 provides a duplicate Western blot analysis of zebrafish treatment groups: A) healthy, untreated; B) diabetic, untreated; C) diabetic, 10 μM rosiglitazone; D) diabetic, 10 μM licarin A (42) or licarin A derivatives 45-48.

FIG. 7 provides Western blot band densities for HbA_{1C} expression in zebrafish treatment groups: A) healthy, untreated; B) diabetic, untreated; C) diabetic, 10 μM rosiglitazone; D) diabetic, 10 μM licarin A (42) or licarin A derivatives 45-48.

FIG. 8 provides Western blot band densities for PPAR-γ expression in zebrafish treatment groups: A) healthy, untreated; B) diabetic, untreated; C) diabetic, 10 μM rosiglitazone; D) diabetic, 10 μM licarin A (42) or licarin A derivatives 45-48.

FIG. 9 provides Western blot band densities for NFκB p65 expression in zebrafish treatment groups: A) healthy, untreated; B) diabetic, untreated; C) diabetic, 10 μM rosiglitazone; D) diabetic, 10 μM licarin A (42) or licarin A derivatives 45-48.

FIG. 10 provides Western blot band densities for PEPCK expression in zebrafish treatment groups: A) healthy, untreated; B) diabetic, untreated; C) diabetic, 10 μM rosiglitazone; D) diabetic, 10 μM licarin A (42) or licarin A derivatives 45-48.

FIG. 11 provides Western blot band densities for GLUT-4 expression in zebrafish treatment groups: A) healthy, untreated; B) diabetic, untreated; C) diabetic, 10 μM rosiglitazone; D) diabetic, 10 μM licarin A (42) or licarin A derivatives 45-48.

FIG. 12 provides the entire extraction scheme of mace as described in the Examples.

Like reference symbols in the various drawings indicate like elements.

### DETAILED DESCRIPTION

The following description of the disclosure is provided as an enabled teaching of the disclosure in its best, currently known embodiments. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are part of the present disclosure. Thus, the following description is provided as illustrative of the principles of the present disclosure and not in limitation thereof.

### Definitions

Unless defined otherwise, all technical and scientific terms used herein have the same meaning as commonly understood to one of ordinary skill in the art to which this invention belongs. The following definitions are provided for the full understanding of terms used in the specification.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g, the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

### Chemical Definitions

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below. As described herein, “perfluoroalkyl” is an alkyl group as described herein where each hydrogen substituent on the group has been substituted with a fluorine atom. Representative but non-limiting examples of “perfluoroalkyl” groups include trifluoromethyl, pentafluoroethyl, or heptadecafluorooctyl.

The symbols A″ is used herein as merely a generic substitutent in the definitions below.

The term “alkoxy” as used herein is an alkyl group bound through a single, terminal ether linkage; that is, an “alkoxy” group can be defined as —OA¹ where A¹ is alkyl as defined above.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl and heteroaryl group can be substituted or unsubstituted. The aryl and heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O) H. Throughout this specification “C(O)” is a short hand notation for C═O.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O⁻.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “nitro” as used herein is represented by the formula —NO₂.

The term “cyano” as used herein is represented by the formula —CN The term “azido” as used herein is repressed by the formula —N₃.

The term “sulfonyl” is used herein to refer to the sulfo-oxo group represented by the formula —S(O)₂A¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH₂.

The term “thiol” as used herein is represented by the formula —SH.

It is to be understood that the compounds provided herein may contain chiral centers. Such chiral centers may be of either the (R—) or (S—) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R—) form is equivalent, for compounds that undergo epimerization in vivo, to administration of the compound in its (S—) form.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

### Compounds

The present disclosure provide compounds that may be useful as agents that selectively targeted pathways, including targeting PPAR, PEPCK, NF-κB, GLUT-4, and PPAR-5/TNKS, and more particularly for the treatment of diabetes or other metabolic disorders.

In one embodiment, a compound of Formula I is provided:

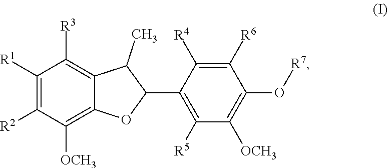

or a pharmaceutically acceptable salt, prodrug, or derivative thereof;
wherein:
R¹ is selected from hydrogen, alkyl, alkenyl, alkynyl, aryl, heteroaryl, cycloalkyl, cycloalkenyl, aldehyde, carboxylic acid, ester, ketone, amino, hydroxy, alkoxy, nitro, and thiol, each of which may be optionally substituted with one or more substituents as allowed by valence independently selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, and thiol;
R², R³, R⁴, R⁵, and R⁶ are each independently selected from hydrogen, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, and thiol, each of which R², R³, R⁴, R⁵, and R⁶ may be optionally substituted with one or more substituents as allowed by valence independently selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol;
R⁷ is selected from hydrogen, alkyl, aryl, heteroaryl, cycloalkyl, C(O)R⁹, P(O)(R⁹)₂, and S(O)₁₋₂R⁹, each of which may be optionally substituted with one or more substituents as allowed by valence independently selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol; and
R⁹ is selected independently at each occurrence from hydrogen, alkyl, halogenated alkyl, alkoxy, alkenyl, alkynyl, aryl, heteroaryl, and amino, each of which R⁹ may be optionally substituted with one or more substituents as allowed by valence independently selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol;
with the proviso that the compound is not:

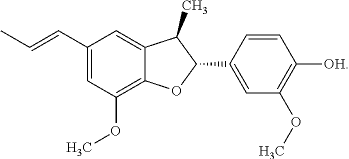 [SMILES: C/C=C/c1cc(OC)c2c(c1)[C@@H](C)[C@H](c1ccc(O)c(OC)c1)O2]

In some embodiments, the compound of Formula I is a compound of Formula Ia:

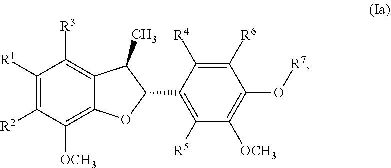

or a pharmaceutically acceptable salt, prodrug, or derivative thereof,
wherein all variables are as defined herein.

In some embodiments, the compound of Formula I is a compound of Formula Ib:

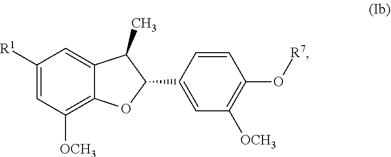

or a pharmaceutically acceptable salt, prodrug, or derivative thereof,
wherein all variables are as defined herein.

In one embodiment, a compound of Formula II is provided:

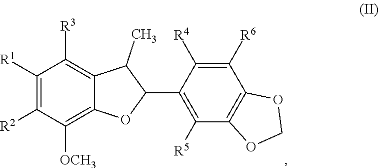

or a pharmaceutically acceptable salt, prodrug, or derivative thereof;
wherein all variables are as defined herein;

with the proviso that the compound of Formula II cannot be:

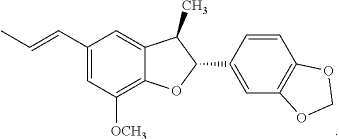 [SMILES: C/C=C/c1cc(OC)c2c(c1)[C@@H](C)[C@H](c1ccc3c(c1)OCO3)O2]

In some embodiments, the compound of Formula II is a compound of Formula IIa:

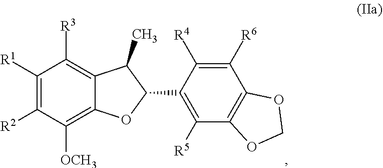

or a pharmaceutically acceptable salt, prodrug, or derivative thereof,
wherein all variables are as defined herein.

In some embodiments, the compound of Formula II is a compound of Formula IIb:

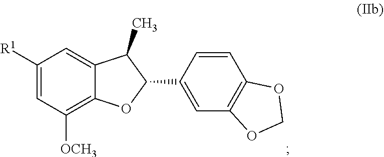

or a pharmaceutically acceptable salt, prodrug, or derivative thereof,
wherein all variables are as defined herein.

In one embodiment, a compound of Formula III is provided:

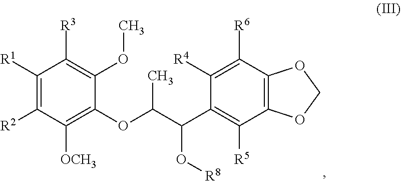

or a pharmaceutically acceptable salt, prodrug, or derivative thereof;
wherein:
R⁸ is selected from hydrogen, alkyl, aryl, heteroaryl, cycloalkyl, C(O)R⁹, P(O)(R⁹)₂, and S(O)₁₋₂R⁹, each of which may be optionally substituted with one or more substituents as allowed by valence independently selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol;
and all other variables are as defined herein;
with the proviso that the compound of formula III cannot be:

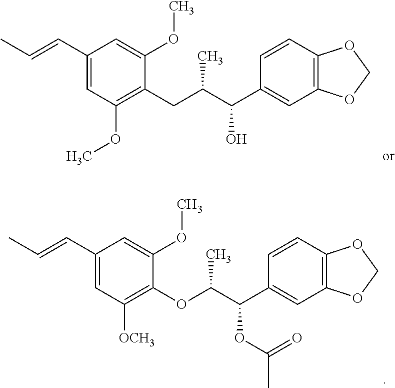 [SMILES: C/C=C/c1cc(OC)c(C[C@H](C)[C@@H](O)c2ccc3c(c2)OCO3)c(OC)c1 ; C/C=C/c1cc(OC)c(O[C@H](C)[C@@H](OC(C)=O)c2ccc3c(c2)OCO3)c(OC)c1]

In some embodiments, the compound of Formula III is a compound of Formula IIIa:

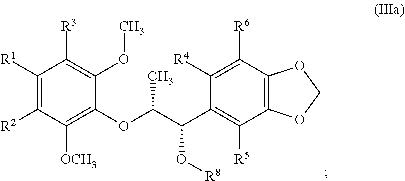

or a pharmaceutically acceptable salt, prodrug, or derivative thereof;
wherein all variables are as defined herein.

In some embodiments, the compound of Formula III is a compound of Formula IIIb;

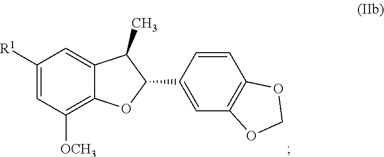

or a pharmaceutically acceptable salt, prodrug, or derivative thereof;
wherein all variables are as defined herein.

In one embodiment, a compound of Formula IV is provided:

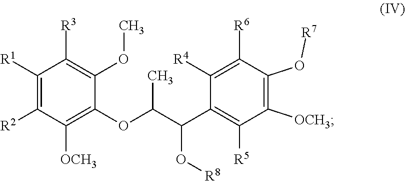

or a pharmaceutically acceptable salt, prodrug, or derivative thereof;
wherein all variables are as defined herein.

In some embodiments, the compound of Formula IV is a compound of Formula IVa:

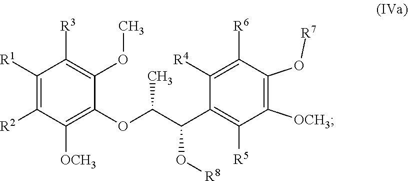

or a pharmaceutically acceptable salt, prodrug, or derivative thereof;
wherein all variables are as defined herein.

In some embodiments, the compound of Formula IV is a compound of Formula IVb:

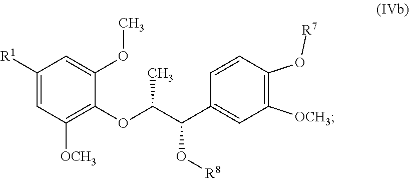

or a pharmaceutically acceptable salt, prodrug, or derivative thereof;
wherein all variables are as defined herein.

In some embodiments, R¹ is C₂-C₆ alkenyl. In some embodiments, R¹ is C₁-C₆ alkyl. In some embodiments, R¹ is C(O) H. In some embodiments, R¹ is C(O)OH. In some embodiments, R¹ is C(O)O(C₁-C₆ alkyl).

In some embodiments, R² is hydrogen. In some embodiments, R² is halogen (for example, fluorine, chlorine, bromine, or iodine). In some embodiments, R² is C₁-C₆ alkyl (for example, methyl).

In some embodiments, R³ is hydrogen. In some embodiments, R³ is halogen (for example, fluorine, chlorine, bromine, or iodine). In some embodiments, R³ is C₁-C₆ alkyl (for example, methyl).

In some embodiments, R⁴ is hydrogen. In some embodiments, R⁴ is halogen (for example, fluorine, chlorine, bromine, or iodine). In some embodiments, R⁴ is C₁-C₆ alkyl (for example, methyl).

In some embodiments, R⁵ is hydrogen. In some embodiments, R⁵ is halogen (for example, fluorine, chlorine, bromine, or iodine). In some embodiments, R⁵ is C₁-C₆ alkyl (for example, methyl).

In some embodiments, R⁶ is hydrogen. In some embodiments, R⁶ is halogen (for example, fluorine, chlorine, bromine, or iodine). In some embodiments, R⁶ is C₁-C₆ alkyl (for example, methyl).

In some embodiments, R⁷ is hydrogen. In some embodiments, R⁷ is C₁-C₆ alkyl (for example, methyl, ethyl, n-propyl, or isopropyl). In some embodiments, R⁷ is C(O)(R⁹).

In some embodiments, R⁸ is hydrogen. In some embodiments, R⁸ is C₁-C₆ alkyl (for example, methyl, ethyl, n-propyl, or isopropyl). In some embodiments, R⁸ is C(O)(R⁹).

In some embodiments, R⁹ is hydrogen. In some embodiments, R⁹ is C₁-C₆ alkyl (for example, methyl, ethyl, n-propyl, or isopropyl). In some embodiments, R⁹ is aryl, for example phenyl. In some embodiments, R⁹ is C₁-C₆ alkoxy, for example methoxy, ethoxy, n-propyloxy, or isopropoxy.

Representative examples of compounds of Formula I include, but are not limited to:

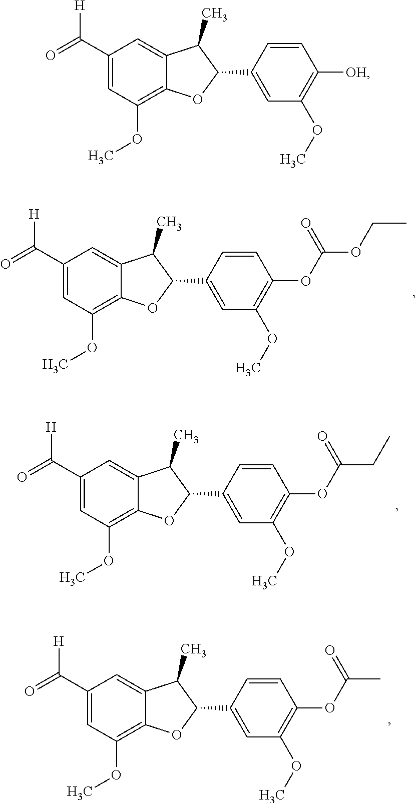 [SMILES: COc1cc([C@@H]2Oc3c(OC)cc(C=O)cc3[C@H]2C)ccc1O ; CCOC(=O)Oc1ccc([C@@H]2Oc3c(OC)cc(C=O)cc3[C@H]2C)cc1OC ; CCC(=O)Oc1ccc([C@@H]2Oc3c(OC)cc(C=O)cc3[C@H]2C)cc1OC ; COc1cc([C@@H]2Oc3c(OC)cc(C=O)cc3[C@H]2C)ccc1OC(C)=O]

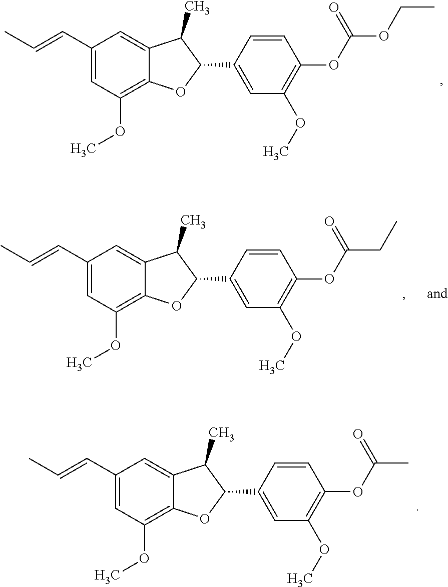

The present disclosure also includes compounds of Formula I, Formula II, Formula III, or Formula IV with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (H) and tritium (³H) may optionally be used anywhere in described structures that achieves the desired result. Alternatively or in addition, isotopes of carbon, e.g., ¹³C and ¹⁴C, may be used. In one embodiment, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tmax, Cmax, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at a desired location. Unless otherwise stated, the enrichment at any point is above natural abundance, and in an embodiment is enough to alter a detectable property of the compounds as a drug in a human.

The compounds of the present disclosure may form a solvate with solvents (including water). Therefore, in one embodiment, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D₂O, d₆-acetone, or d₆-DMSO. A solvate can be in a liquid or solid form.

A “prodrug” as used herein means a compound which when administered to a host in vivo is converted into a parent drug. As used herein, the term “parent drug” means the presently described compound herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain embodiments, the prodrug renders the parent compound more lipophilic. In certain embodiments, a prodrug can be provided that has several prodrug moieties in a linear, branched, or cyclic manner. For example, non-limiting embodiments include the use of a divalent linker moiety such as a dicarboxylic acid, amino acid, diamine, hydroxycarboxylic acid, hydroxyamine, di-hydroxy compound, or other compound that has at least two functional groups that can link the parent compound with another prodrug moiety, and is typically biodegradable in vivo. In some embodiments, 2, 3, 4, or 5 prodrug biodegradable moieties are covalently bound in a sequence, branched, or cyclic fashion to the parent compound. Non-limiting examples of prodrugs according to the present disclosure are formed with: a hydroxyl group on the parent drug and a carboxylic acid on the prodrug moiety to form an ester; a hydroxyl on the parent drug and a hydroxylated prodrug moiety to form an ester; a hydroxyl on the parent drug and a phosphonate on the prodrug to form a phosphonate ester; a hydroxyl on the parent drug and a phosphoric acid prodrug moiety to form a phosphate ester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—S—(C₂₋₂₄ alkyl) to form an thioether; and a hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some embodiments, a prodrug is provided by attaching a natural or non-natural amino acid to an appropriate functional moiety on the parent compound, for example, oxygen, usually in a manner such that the amino acid is cleaved in vivo to provide the parent drug. The amino acid can be used alone or covalently linked (straight, branched or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Methods of Treatment

The present disclosure also provides methods for the treatment of a disease or disorder in a subject in need thereof that can be treated by modulating the activity of PPAR-γ, NFκB, GLUT-4, and/or PEPCK. In some embodiments, the disease or disorder is a metabolic disorder. In some embodiments, a method for ameliorating one or more symptoms of a metabolic disorder is provided comprising administering a therapeutically effective amount of a compound described herein, or a pharmaceutically acceptable salt, prodrug, or derivative thereof. In some embodiments, a method for reducing the severity of one or more symptoms of a metabolic disorder is provided comprising administering a therapeutically effective amount of a compound described herein, or a pharmaceutically acceptable salt, prodrug, or derivative thereof.

In some embodiments, the metabolic disorder may be selected from diabetes, obesity, metabolic syndrome, diabetic dyslipidemia, or hypertriglyceridemia. In some embodiments, the metabolic disorder may be selected from a fatty liver disease, dyslipidemia, metabolic syndrome, a cardiovascular disease, obesity, a leptin disorder, or any combination thereof. In some embodiments, the fatty liver disease may be selected from hepatic steatosis, non-alcoholic steatohepatitis, non-alcoholic fatty liver disease, elevated liver cholesterol level, or elevated liver non-HDL cholesterol level, or any combination thereof. In some embodiments, dyslipidemia may be selected from hyperlipidemia, mixed dyslipidemia, hypercholesterolemia, polygenic hypercholesterolemia, hypertriglyceridemia, hyperfattyacidemia, elevated ApoB, elevated cholesterol, elevated LDL-cholesterol, elevated VLDL-cholesterol, or elevated non-HDL-cholesterol, or any combination thereof. In some embodiments, the cardiovascular disease may include coronary heart disease, acute coronary syndrome, early onset coronary heart disease, or atherosclerosis, or any combination thereof. In some embodiments, the leptin disorder may include lyperleptinemia, or tissue leptin resistance, or a combination thereof.

In one embodiment, a method for treating diabetes is provided comprising administering a therapeutically effective amount of a compound described herein, or a pharmaceutically acceptable salt, prodrug, or derivative thereof.

In one embodiment, a method for treating inflammation is provided comprising administering a therapeutically effective amount of a compound described herein, or a pharmaceutically acceptable salt, prodrug, or derivative thereof.

In some embodiments, the methods described herein can be used to treat inflammation caused by: a physical cause such as burns, frostbite, physical injury (either blunt or penetrating), foreign bodies (including splinters, dirt, or debris), trauma, or ionizing radiation; a biological cause such as infection by a pathogen, an immune reaction due to hypersensitivity, or stress; or a chemical cause such as a chemical irritant, a toxin.

In some embodiments, the inflammation comprises acute inflammation. In some embodiments, the acute inflammation may be in response to one or more of the following: a wound (such as a cut, bruise, or burn); an infection (such as a bacterial, viral, fungal, or protist infection); exposure to a toxin or ionizing radiation; exposure to an allergen or antigen; and the presence of a foreign body (for example, a splinter) in a subject.

In some embodiments, the inflammation comprises chronic inflammation. In some embodiments, the chronic inflammation may be associated with a persistent form of acute inflammation, as described above, or may be associated with an inflammatory disorder.

The present methods may be used to treat or prevent inflammation in any part of the body, including but not limited to inflammation of: the central nervous system (such as encephalitis, myelitis, or meningitis); the peripheral nervous system (such as neuritis); the eye (such as dacryoadenitis, scleritis, episcleritis, or keratitis); the ear (such as otitis); the heart (such as endocarditis, myocarditis, or pericarditis); the vascular system (such as arteritis, phlebitis, or capillaritis); the respiratory system (such as sinusitis, rhinitis, pharyngitis, epiglottitis, laryngitis, tracheitis, bronchitis, pneumonitis, or pleurisy); the digestive system (such as stomatitis, gingivitis, glossitis, tonsillitis, sialadenitis, parotitis, cheilitis, pulpitis, gnathitis, oesophagitis, gastritis, gastroenteritis, enteritis, colitis, pancolitis, appendicitis, cryptitis, hepatitis, cholecystitis, or pancreatitis); the integumentary system (such as dermatitis or mastitis); the musculoskeletal system (such as arthritis, myositis, synovitis, tenosynovitis, or bursitis); the urinary system (such as nephritis, ureteritis, cystitis, or urethritis); the female reproductive system (such as oophoritis, salpingitis, endometritis, myometritis, parametritis, cervicitis, vaginitis, or vulvitis); the male reproductive system (such as orchitis, epididymitis, prostatitis, vasculitis, balanitis, or posthitis); the endocrine system (such as insulitis, hypophysitis, thyroiditis, parathyroiditis, or adrenalitis); or the lymphatic system (such a lymphangitis or lymphadenitis).

The present methods may also be used to treat or prevent inflammation resulting from an inflammatory disorder. In some embodiments, the methods described herein may be used as an analgesic to treat pain, for example a headache. In some embodiments, the methods described herein may be used to treat arthritis, including but not limited to rheumatoid arthritis, spondyloarthopathies, gouty arthritis, systemic lupus erythematosus, osteoarthritis, and juvenile arthritis. In some embodiments, the methods described herein may be used to treat asthma, bronchitis, menstrual cramps, tendinitis, bursitis, and skin related conditions such as psoriasis, eczema, burns and dermatitis. In some embodiments, the methods described herein may be used to treat gastrointestinal conditions such as inflammatory bowel disease, Crohn's disease, gastritis, irritable bowel syndrome, and ulcerative colitis. In some embodiments, the methods described herein may be used to treat inflammation present in a disorder including, but not limited to, vascular disease, migraine headaches, perarteritis nodosa, thyroiditis, aplastic anemia, Hodgkin's disease, scleroderma, rheumatic fever, type I diabetes, myasthenia gravis, sarcoidosis, nephrotic syndrome, Behcet's syndrome, polymyositis, hypersensitivity, conjunctivitis, gingivitis, swelling occurring after an injury, myocardial ischemia, and the like.

In some embodiments, the methods described herein may be used to treat or prevent inflammation associated with a disorder including, but not limited to, acne vulgaris, asthma, an autoimmune disease, an autoinflammatory disease, celiac disease, chronic prostatitis, colitis, diverticulitis, glomerulonephritis, hidradenitis suppurativa, hypersensitivities, inflammatory bowel disease, interstitial cystitis, lichen planus, mast cell activation syndrome, otitis, pelvic inflammatory disease, reperfusion injury, rheumatic fever, rheumatoid arthritis, rhinitis, sarcoidosis, transplant rejection, or vasculitis. In some embodiments, the methods described herein may be used to treat or prevent inflammation associated with atherosclerosis, cancer, or ischemic heart disease.

In some embodiments, the methods described herein may be used to treat a systemic inflammatory disorder or ameliorate or diminish one or more inflammatory symptoms of a system inflammatory disorder including, but not limited to, non-alcoholic fatty liver disease, non-alcoholic steatohepatitis, inflammatory bowel disease, Crohn's disease, ulcerative colitis, psoriasis, irritable bowel syndrome, ankylosing spondylitis, osteoporosis, rheumatoid arthritis, psoriatic arthritis, chronic obstructive pulmonary disease, atherosclerosis, pulmonary arterial hypertension, pyridoxine-dependent epilepsy, atopic dermatitis, rosacea, multiple sclerosis, systemic lupus erythematosus, lupus nephritis, sepsis, eosinophilic esophagitis, chronic kidney disease, fibrotic renal disease, chronic eosinophilic pneumonia, extrinsic allergic alveolitis, pre-eclampsia, endometriosis, polycystic ovary syndrome, or cyclophosphamide-induced hemorrhagic cystitis.

In some embodiments, the methods described herein may be used to treat inflammation resulting from a disorder selected from light chain deposition disease, IgA nephropathy, end-stage renal disease, gout, pseudogout, diabetic nephropathy, diabetic neuropathy, traumatic brain injury, noise-induced hearing loss, Alzheimer's disease, Parkinson's disease, Huntington disease, amyotrophic lateral sclerosis, primary biliary cirrhosis, primary sclerosing cholangitis, uterine leiomyoma, sarcoidosis, or chronic kidney disease.

In another aspect, a method of treating a metabolic disorder in a subject in need thereof, as described herein, is provided comprising administering a therapeutically effective amount of a compound selected from a compound of Formula A, Formula B, Formula C, or Formula D:

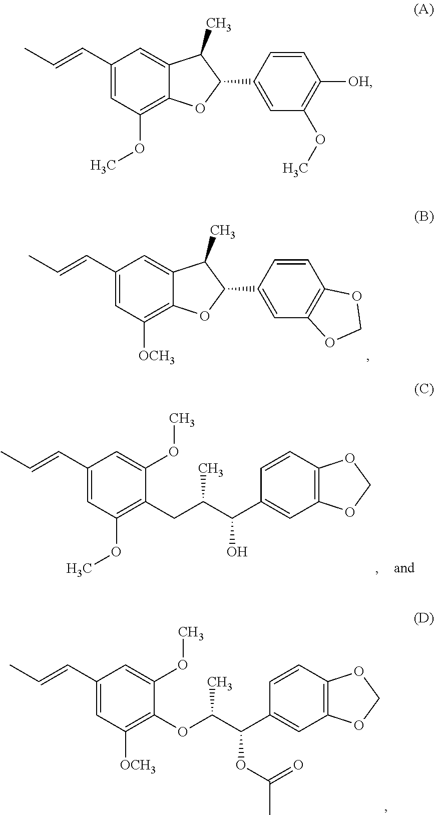 [SMILES: C/C=C/c1cc(OC)c2c(c1)[C@@H](C)[C@H](c1ccc(O)c(OC)c1)O2 ; C/C=C/c1cc(OC)c2c(c1)[C@@H](C)[C@H](c1ccc3c(c1)OCO3)O2 ; C/C=C/c1cc(OC)c(C[C@H](C)[C@@H](O)c2ccc3c(c2)OCO3)c(OC)c1 ; C/C=C/c1cc(OC)c(O[C@H](C)[C@@H](OC(C)=O)c2ccc3c(c2)OCO3)c(OC)c1]

or a pharmaceutically acceptable salt, prodrug, or derivative thereof.

In another aspect, a method of treating inflammation in a subject in need thereof, as described herein, is provided comprising administering a therapeutically effective amount of a compound selected from a compound of Formula A, Formula B, Formula C, or Formula D, or a pharmaceutically acceptable salt, prodrug, or derivative thereof.

### Methods of Administration

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and an excipient of some sort may be useful in a variety of medical and non-medical applications. For example, pharmaceutical compositions comprising an active compound and an excipient may be useful for the treatment or prevention of a disease or disorder, for example a metabolic disorder such as diabetes.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, tale, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy (Polyethylene glycol)-1000], 1,2-Distearoyl-sn-glycero-3-Phosphoethanolamine-N-[Methoxy (Polyethylene glycol)-2000], and 1,2-Distearoyl-sn-glycero-3-Phosphoethanolamine-N-[Methoxy (Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the disorder, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed, the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Additional embodiments of the present disclosure are provided below:

Embodiment 1. A compound of Formula I, Formula II, Formula III, or Formula IV:

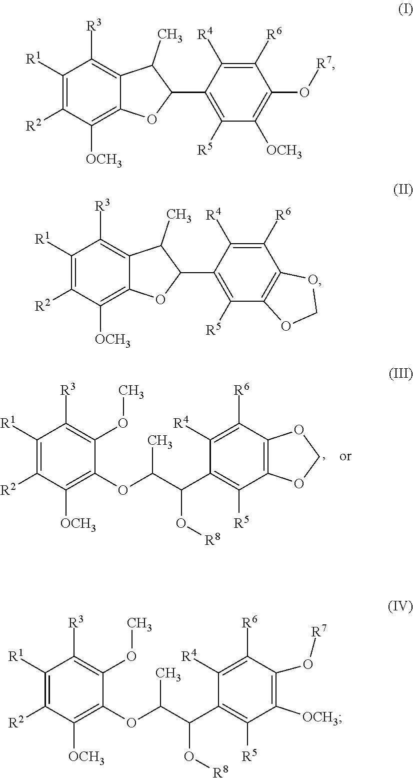

or a pharmaceutically acceptable salt, prodrug, or derivative thereof;
wherein:
R¹ is selected from hydrogen, alkyl, alkenyl, alkynyl, aryl, heteroaryl, cycloalkyl, cycloalkenyl, aldehyde, carboxylic acid, ester, ketone, amino, hydroxy, alkoxy, nitro, and thiol, each of which may be optionally substituted with one or more substituents as allowed by valence independently selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, and thiol;
R², R³, R⁴, R⁵, and R⁶ are each independently selected from hydrogen, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, and thiol, each of which R², R³, R⁴, R⁵, and R⁶ may be optionally substituted with one or more substituents as allowed by valence independently selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol;
R⁷ is selected from hydrogen, alkyl, aryl, heteroaryl, cycloalkyl, C(O)R⁹, P(O)(R⁹)₂, and S(O)₁₋₂R⁹, each of which may be optionally substituted with one or more substituents as allowed by valence independently selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol;
R⁸ is selected from hydrogen, alkyl, aryl, heteroaryl, cycloalkyl, C(O)R⁹, P(O)(R⁹)₂, and S(O)₁₋₂R⁹, each of which may be optionally substituted with one or more substituents as allowed by valence independently selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol; and
R⁹ is selected independently at each occurrence from hydrogen, alkyl, halogenated alkyl, alkoxy, alkenyl, alkynyl, aryl, heteroaryl, and amino, each of which R⁹ may be optionally substituted with one or more substituents as allowed by valence independently selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol;
with the proviso that the compound of Formula I cannot be:

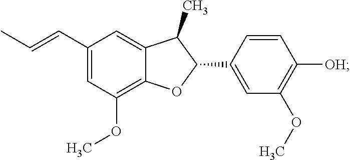 [SMILES: C/C=C/c1cc(OC)c2c(c1)[C@@H](C)[C@H](c1ccc(O)c(OC)c1)O2]

the compound of Formula II cannot be:

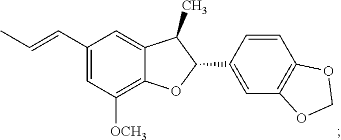 [SMILES: C/C=C/c1cc(OC)c2c(c1)[C@@H](C)[C@H](c1ccc3c(c1)OCO3)O2]

and
the compound of formula III cannot be:

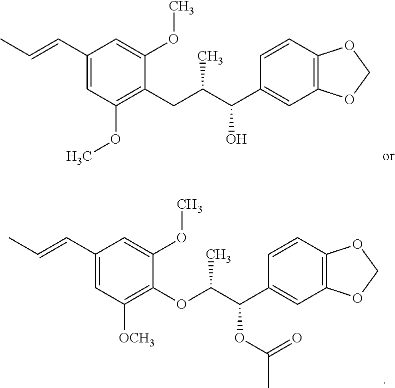 [SMILES: C/C=C/c1cc(OC)c(C[C@H](C)[C@@H](O)c2ccc3c(c2)OCO3)c(OC)c1 ; C/C=C/c1cc(OC)c(O[C@H](C)[C@@H](OC(C)=O)c2ccc3c(c2)OCO3)c(OC)c1]

Embodiment 2. The compound of embodiment 1, wherein the compound is of Formula Ia:

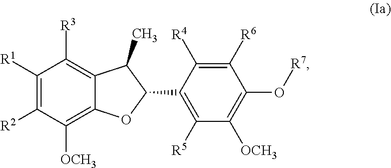

or a pharmaceutically acceptable salt, prodrug, or derivative thereof.

Embodiment 3. The compound of embodiment 1, wherein the compound is of Formula IIa:

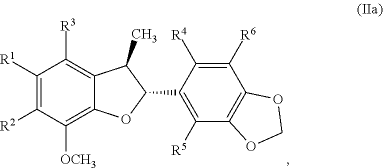

or a pharmaceutically acceptable salt, prodrug, or derivative thereof.

Embodiment 4. The compound of embodiment 1, wherein the compound is of Formula IIIa:

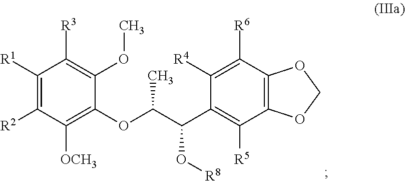

or a pharmaceutically acceptable salt, prodrug, or derivative thereof.

Embodiment 5. The compound of embodiment I, wherein the compound is of Formula IVa:

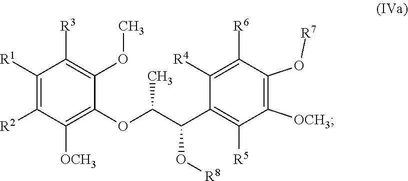

or a pharmaceutically acceptable salt, prodrug, or derivative thereof.

Embodiment 6. The compound of any one of embodiments 1 or 2, wherein the compound is of Formula Ib:

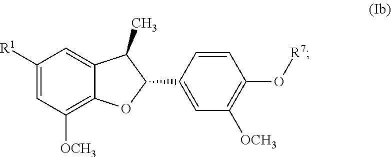

or a pharmaceutically acceptable salt, prodrug, or derivative thereof.

Embodiment 7. The compound of any one of embodiments 1 or 3, wherein the compound is of Formula IIb:

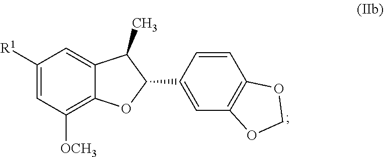

or a pharmaceutically acceptable salt, prodrug, or derivative thereof.

Embodiment 8. The compound of any one of embodiments 1 or 4, wherein the compound is of Formula IIIb:

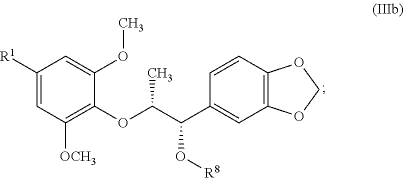

or a pharmaceutically acceptable salt, prodrug, or derivative thereof.

Embodiment 9. The compound of any one of embodiments 1 or 5, wherein the compound is of Formula IVb:

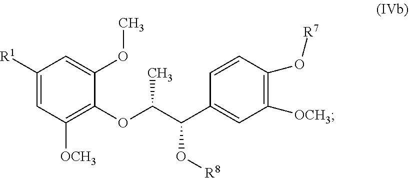

or a pharmaceutically acceptable salt, prodrug, or derivative thereof.

Embodiment 10. The compound of any one of embodiments 1-5, wherein R², R³, R⁴, R⁵, and Re are each independently selected from hydrogen, halogen, or C₁-C₆ alkyl.

Embodiment 11. The compound of embodiment 10, wherein R² is selected from hydrogen, fluoro, chloro, bromo, iodo, and methyl.

Embodiment 12. The compound of embodiment 10 or embodiment 11, wherein R³ is selected from hydrogen, fluoro, chloro, bromo, iodo, and methyl.

Embodiment 13. The compound of any one of embodiments 10-12, wherein R⁴ is selected from hydrogen, fluoro, chloro, bromo, iodo, and methyl.

Embodiment 14. The compound of any one of embodiments 10-13, wherein R⁵ is selected from hydrogen, fluoro, chloro, bromo, iodo, and methyl.

Embodiment 15. The compound of any one of embodiments 10-14, wherein R⁶ is selected from hydrogen, fluoro, chloro, bromo, iodo, and methyl.

Embodiment 16. The compound of any one of embodiments 1, 2, 5, 6, 9, and 10-15, wherein R¹ is selected from C₁-C₆ alkyl and C(O)R⁹.

Embodiment 17. The compound of embodiment 16, wherein R⁷ is selected from methyl, ethyl, n-propyl, or isopropyl.

18. The compound of embodiment 16, wherein R⁷ is C(O)(R⁹), wherein R⁹ is C₁-C₆ alkyl, C₁-C₆ alkoxy, or aryl.

Embodiment 19. The compound of embodiment 16, wherein R⁷ is C(O)(R⁹), wherein R⁹ is methyl, ethyl, n-propyl, isopropyl, phenyl, methoxy, ethoxy, n-propoxy, and isopropoxy.

Embodiment 20. The compound of any one of embodiments 1, 4, 5, and 8-19, wherein R⁸ is selected from C₁-C₆ alkyl and C(O)R⁹.

Embodiment 21. The compound of embodiment 20, wherein R& is selected from methyl, ethyl, n-propyl, or isopropyl.

Embodiment 22. The compound of embodiment 20, wherein RR is C(O)(R⁹), wherein R⁹ is C₁-C₆ alkyl, C₁-C₆ alkoxy, or aryl.

Embodiment 23. The compound of embodiment 20, wherein R⁸ is C(O)(R⁹), wherein R⁹ is methyl, ethyl, n-propyl, isopropyl, phenyl, methoxy, ethoxy, n-propoxy, and isopropoxy.

Embodiment 24. The compound of any one of embodiments 1-23, wherein R¹ is selected from C₂-C₆ alkenyl and C(O) H.

Embodiment 25. The compound of embodiment 1, wherein the compound is selected from:

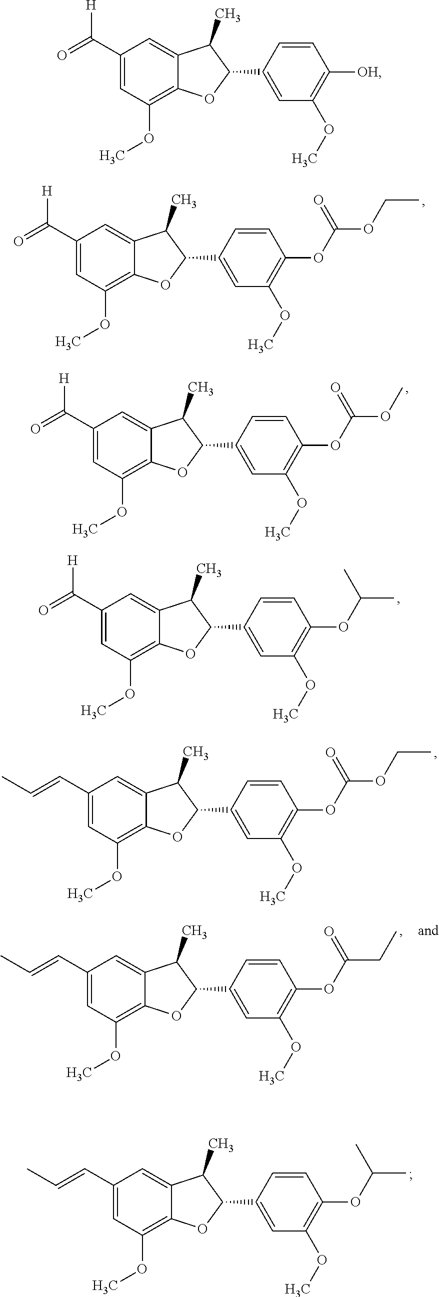

or a pharmaceutically acceptable salt, prodrug, or derivative thereof.

Embodiment 26. A pharmaceutical composition comprising a compound of any one of embodiments 1-25, or a pharmaceutically acceptable salt, prodrug, or derivative thereof, in a pharmaceutically acceptable carrier.

Embodiment 27. A method of treating a method of treating a metabolic disorder in a subject in need thereof comprising administering a therapeutically effective amount of a compound of any one of embodiments 1-25 to the subject, or a pharmaceutically acceptable salt, prodrug, or derivative thereof, or a pharmaceutical composition of embodiment 26.

Embodiment 28. The method of embodiment 27, wherein the metabolic disorder is diabetes.

Embodiment 29. The method of embodiment 27, wherein the metabolic disorder is obesity.

Embodiment 30. The method of embodiment 27, wherein the metabolic disorder is metabolic syndrome.

Embodiment 31. The method of embodiment 27, wherein the metabolic disorder is diabetic dyslipidemia.

Embodiment 32. The method of embodiment 27, wherein the metabolic disorder is bypertriglyceridemia.

Embodiment 33. A method of treating hyperglycemia in a subject in need thereof comprising administering a therapeutically effective amount of a compound of any one of embodiments 1-25 to the subject, or a pharmaceutically acceptable salt, prodrug, or derivative thereof, or a pharmaceutical composition of embodiment 26.

Embodiment 34. A method of treating inflammation in a subject in need thereof comprising a therapeutically effective amount of a compound of any one of embodiments 1-25 to the subject, or a pharmaceutically acceptable salt, prodrug, or derivative thereof, or a pharmaceutical composition of embodiment 26.

Embodiment 35. The method of embodiment 34, wherein the inflammation is acute inflammation.

Embodiment 36. The method of embodiment 34, wherein the inflammation is chronic inflammation.

Embodiment 37. The method of embodiment 34, wherein the inflammation is associated with an inflammatory disorder.

Embodiment 38. The method of embodiment 34, wherein the inflammation is selected from encephalitis, myelitis, meningitis, neuritis, dacryoadenitis, scleritis, episcleritis, keratitis, otitis, endocarditis, myocarditis, pericarditis, arteritis, phlebitis, capillaritis, sinusitis, rhinitis, pharyngitis, epiglottitis, laryngitis, tracheitis, bronchitis, pneumonitis, pleurisy, stomatitis, gingivitis, glossitis, tonsillitis, sialadenitis, parotitis, cheilitis, pulpitis, gnathitis, oesophagitis, gastritis, gastroenteritis, enteritis, colitis, pancolitis, appendicitis, cryptitis, hepatitis, cholecystitis, pancreatitis, dermatitis, mastitis, arthritis, myositis, synovitis, tenosynovitis, bursitis, nephritis, ureteritis, cystitis, urethritis, oophoritis, salpingitis, endometritis, myometritis, parametritis, cervicitis, vaginitis, vulvitis, orchitis, epididymitis, prostatitis, vasculitis, balanitis, posthitis, insulitis, hypophysitis, thyroiditis, parathyroiditis, adrenalitis, lymphangitis and lymphadenitis.

Embodiment 39. The method of embodiment 34, wherein the inflammation is associated with arthritis.

Embodiment 40. The method of embodiment 39, wherein the arthritis is selected from rheumatoid arthritis, spondyloarthopathies, gouty arthritis, systemic lupus erythematosus, osteoarthritis, and juvenile arthritis.

Embodiment 41. The method of embodiment 34, wherein the inflammation is associated with asthma, bronchitis, menstrual cramps, tendinitis, bursitis, psoriasis, eczema, burns or dermatitis.

Embodiment 42. The method of embodiment 34, wherein the inflammation is associated with a gastrointestinal condition.

Embodiment 43. The method of embodiment 42, wherein the gastrointestinal condition is selected from inflammatory bowel disease, Crohn's disease, gastritis, irritable bowel syndrome, and ulcerative colitis.

Embodiment 44 The method of embodiment 34, wherein the inflammation is associated with vascular disease, migraine headaches, perarteritis nodosa, thyroiditis, aplastic anemia, Hodgkin's disease, scleroderma, rheumatic fever, type I diabetes, myasthenia gravis, sarcoidosis, nephrotic syndrome, Behcet's syndrome, polymyositis, hypersensitivity, conjunctivitis, gingivitis, swelling occurring after an injury, or myocardial ischemia.

Embodiment 45. The method of embodiment 34, wherein the inflammation is associated with a systemic inflammatory disorder.

Embodiment 46. The method of embodiment 45, wherein the systemic inflammatory disorder is selected from non-alcoholic fatty liver disease, non-alcoholic steatohepatitis, inflammatory bowel disease, Crohn's disease, ulcerative colitis, psoriasis, irritable bowel syndrome, ankylosing spondylitis, osteoporosis, rheumatoid arthritis, psoriatic arthritis, chronic obstructive pulmonary disease, atherosclerosis, pulmonary arterial hypertension, pyridoxine-dependent epilepsy, atopic dermatitis, rosacea, multiple sclerosis, systemic lupus erythematosus, lupus nephritis, sepsis, eosinophilic esophagitis, chronic kidney disease, fibrotic renal disease, chronic eosinophilic pneumonia, extrinsic allergic alveolitis, pre-eclampsia, endometriosis, polycystic ovary syndrome, and cyclophosphamide-induced hemorrhagic cystitis.

Embodiment 47. A method of treating a metabolic disorder in a subject in need thereof comprising administering a therapeutically effective amount of a compound selected from a compound of Formula A, Formula B, Formula C, or Formula D:

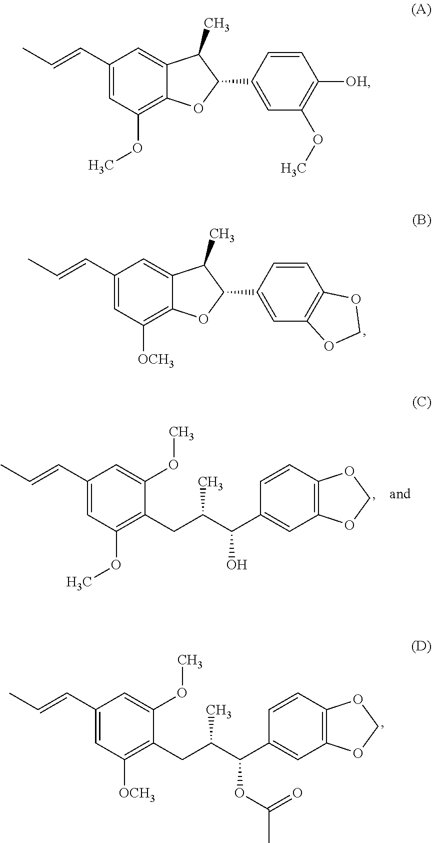 [SMILES: C/C=C/c1cc(OC)c2c(c1)[C@@H](C)[C@H](c1ccc(O)c(OC)c1)O2 ; C/C=C/c1cc(OC)c2c(c1)[C@@H](C)[C@H](c1ccc3c(c1)OCO3)O2 ; C/C=C/c1cc(OC)c(C[C@H](C)[C@@H](O)c2ccc3c(c2)OCO3)c(OC)c1 ; C/C=C/c1cc(OC)c(C[C@H](C)[C@@H](OC(C)=O)c2ccc3c(c2)OCO3)c(OC)c1]

or a pharmaceutically acceptable salt, prodrug, or derivative thereof.

Embodiment 48. The method of embodiment 47, wherein the metabolic disorder is diabetes.

Embodiment 49. The method of embodiment 47, wherein the metabolic disorder is obesity.

Embodiment 50. The method of embodiment 47, wherein the metabolic disorder is metabolic syndrome.

Embodiment 51. The method of embodiment 47, wherein the metabolic disorder is diabetic dyslipidemia.

Embodiment 52. The method of embodiment 47, wherein the metabolic disorder is hypertriglyceridemia.

Embodiment 53. A method of treating inflammation in a subject in need thereof comprising administering a therapeutically effective amount of a compound selected from a compound of Formula A, Formula B, Formula C, or Formula D:

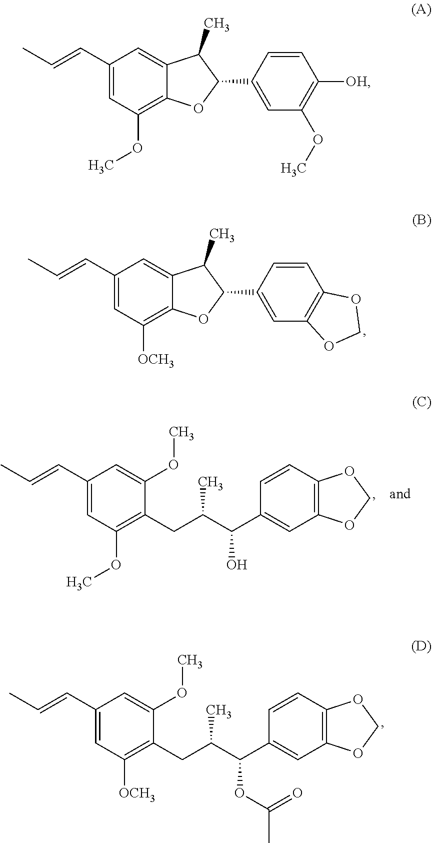 [SMILES: C/C=C/c1cc(OC)c2c(c1)[C@@H](C)[C@H](c1ccc(O)c(OC)c1)O2 ; C/C=C/c1cc(OC)c2c(c1)[C@@H](C)[C@H](c1ccc3c(c1)OCO3)O2 ; C/C=C/c1cc(OC)c(C[C@H](C)[C@@H](O)c2ccc3c(c2)OCO3)c(OC)c1 ; C/C=C/c1cc(OC)c(C[C@H](C)[C@@H](OC(C)=O)c2ccc3c(c2)OCO3)c(OC)c1]

or a pharmaceutically acceptable salt, prodrug, or derivative thereof.

Embodiment 54. The method of embodiment 53, wherein the inflammation is acute inflammation.

Embodiment 55. The method of embodiment 53, wherein the inflammation is chronic inflammation.

Embodiment 56. The method of embodiment 53, wherein the inflammation is associated with an inflammatory disorder.

Embodiment 57. The method of embodiment 53, wherein the inflammation is selected from encephalitis, myelitis, meningitis, neuritis, dacryoadenitis, scleritis, episcleritis, keratitis, otitis, endocarditis, myocarditis, pericarditis, arteritis, phlebitis, capillaritis, sinusitis, rhinitis, pharyngitis, epiglottitis, laryngitis, tracheitis, bronchitis, pneumonitis, pleurisy, stomatitis, gingivitis, glossitis, tonsillitis, sialadenitis, parotitis, cheilitis, pulpitis, gnathitis, oesophagitis, gastritis, gastroenteritis, enteritis, colitis, pancolitis, appendicitis, cryptitis, hepatitis, cholecystitis, pancreatitis, dermatitis, mastitis, arthritis, myositis, synovitis, tenosynovitis, bursitis, nephritis, ureteritis, cystitis, urethritis, oophoritis, salpingitis, endometritis, myometritis, parametritis, cervicitis, vaginitis, vulvitis, orchitis, epididymitis, prostatitis, vasculitis, balanitis, posthitis, insulitis, hypophysitis, thyroiditis, parathyroiditis, adrenalitis, lymphangitis and lymphadenitis.

Embodiment 58. The method of embodiment 53, wherein the inflammation is associated with arthritis.

Embodiment 59. The method of embodiment 58, wherein the arthritis is selected from rheumatoid arthritis, spondyloarthopathies, gouty arthritis, systemic lupus erythematosus, osteoarthritis, and juvenile arthritis.

Embodiment 60. The method of embodiment 53, wherein the inflammation is associated with asthma, bronchitis, menstrual cramps, tendinitis, bursitis, psoriasis, eczema, burns or dermatitis.

Embodiment 61. The method of embodiment 53, wherein the inflammation is associated with a gastrointestinal condition.

Embodiment 62. The method of embodiment 61, wherein the gastrointestinal condition is selected from inflammatory bowel disease, Crohn's disease, gastritis, irritable bowel syndrome, and ulcerative colitis.

Embodiment 63. The method of embodiment 53, wherein the inflammation is associated with vascular disease, migraine headaches, perarteritis nodosa, thyroiditis, aplastic anemia, Hodgkin's disease, scleroderma, rheumatic fever, type I diabetes, myasthenia gravis, sarcoidosis, nephrotic syndrome, Behcet's syndrome, polymyositis, hypersensitivity, conjunctivitis, gingivitis, swelling occurring after an injury, or myocardial ischemia.

Embodiment 64. The method of embodiment 53, wherein the inflammation is associated with a systemic inflammatory disorder.

Embodiment 65. The method of embodiment 64, wherein the systemic inflammatory disorder is selected from non-alcoholic fatty liver disease, non-alcoholic steatohepatitis, inflammatory bowel disease, Crohn's disease, ulcerative colitis, psoriasis, irritable bowel syndrome, ankylosing spondylitis, osteoporosis, rheumatoid arthritis, psoriatic arthritis, chronic obstructive pulmonary disease, atherosclerosis, pulmonary arterial hypertension, pyridoxine-dependent epilepsy, atopic dermatitis, rosacea, multiple sclerosis, systemic lupus erythematosus, lupus nephritis, sepsis, eosinophilic esophagitis, chronic kidney disease, fibrotic renal disease, chronic eosinophilic pneumonia, extrinsic allergic alveolitis, pre-eclampsia, endometriosis, polycystic ovary syndrome, and cyclophosphamide-induced hemorrhagic cystitis.

A number of embodiments of the disclosure have been described. Nevertheless, it will be understood that various modifications may be made without departing from the spirit and scope of the invention. Accordingly, other embodiments are within the scope of the following claims.

### Examples

The following examples are put forth to provide those of ordinary skill in the art with a complete disclosure and description of how the compounds, compositions, and methods claimed herein are made and evaluated and are intended to be purely exemplary of the invention and are not intended to limit the scope of what the inventors regard as their invention. Efforts have been made to ensure accuracy concerning numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

### Experimental Procedures

### General Experimental Methods

Open vacuum and flash column chromatography were performed using 60 Å high purity grade silica gel (70-230 and 230-400 mesh, respectively) from Sigma-Aldrich Co. LLC (St. Louis, MO, USA). To monitor the separations, fractions were chromatographed on TLC plates developed using a solution of 10% sulfuric acid in ethanol (v/v) with 1% vanillin (w/v), and heating the plates to approximately 120° C.

Preparative HPLC was performed using a Waters 600 controller pump and 996 photodiode array detector All HPLC separations for mace natural products were carried out on a SunFire RP18 column (10×150 mm, 5 μm) from Waters (Milford, MA, USA) using a 3.0 mL/min flow rate. HPLC separations of mace derivatives were carried out on the above-mentioned SunFire column or a Varian Polaris C₁₈-A column (21.2×250 mm, 10 μm) from Agilent Technologies (Santa Clara, CA, USA), which was used with a 7.0 mL/min flow rate.

¹H, ¹³C, DEPT. HSQC, and HMBC NMR spectra were obtained using a Bruker Avance NEO 400 MHz spectrometer. The ¹H NMR spectra were recorded at 400 MHz with tetramethylsilane (TMS) as an internal standard and chloroform-d (CDCl₃), methanol-d₄ (CD₃OD), or dimethyl sulfoxide-de as solvents (Cambridge Isotope Laboratories, MA, USA). All NMR data were collected at 300 K.

Molecular weights were obtained using a 15T Bruker SolariX XR Fourier-transform ion cyclotron resonance mass spectrometer (Bruker Scientific, LLC) using HRESI in the positive-ion mode.

Specific rotation of pure compounds was determined using the Anton Paar MCP 150 polarimeter (Anton Paar USA Inc., Ashland, VA, USA). The isolated compounds were characterized and identified by comparing their physical properties and spectroscopic data with literature reports (MS, ¹H NMR, and ¹³C NMR).

### Bioactivity-Guided Isolation

### Plant Material

Dried, whole arils of M. fragrans (mace) were purchased at an Indian grocery. A voucher specimen was deposited in the laboratory with the code D12101 used to identify the source material, the project assignment, and the date the investigation was initiated. The plant species was identified as M. fragrans by the common name “mace” on the product packaging label and by visual comparison to images of M. fragrans arils published in Van Gils and Cox, 1994.324 Circumstantial evidence further supporting the species identification included the dry but leathery texture of the arils, as well as a more delicate version of the distinct nutmeg fragrance.

Solvent Extraction and Partitioning of Myristica fragrans

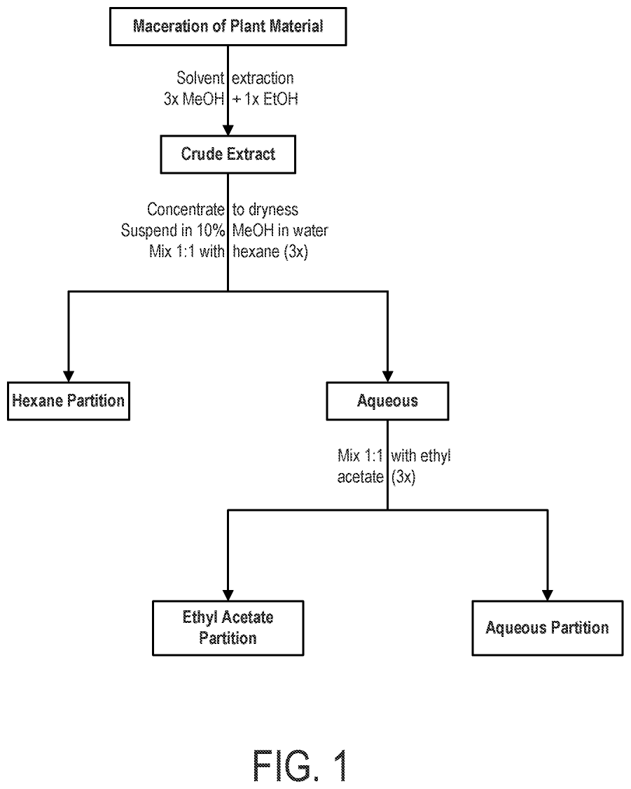

Dried, whole arils from M. fragrans (1.3 kg) were macerated in 2 L methanol for 24 hours and extracted three times. The filtrates were collectively concentrated under vacuum to yield the crude extract, which was dissolved using 10% methanol in water and subjected to solvent-solvent partitioning (3×300 mL) with hexane and ethyl acetate according to FIG. 1. Extract and partition yields are recorded in Table 1. The partitions were subjected to the panel of antidiabetic in vitro assays for bioassay-guided fractionation, and the ethyl acetate partition (D12101-D001) was selected for further investigation.

TABLE 1
Mace extract and partition yields

| Sample | Code | Mass (g) |
|---|---|---|
| Dry Material | D12101 | 1300 |
| Methanol Extract | | 298 |
| Hexane Partition | | 114 |
| Ethyl Acetate Partition | D12101-D001 | 157.5 |
| Aqueous Partition | | 26.4 |

Fractionation of M. fragrans Ethyl Acetate Partition

The ethyl acetate partition (107.8 g) was separated by open vacuum low-pressure column chromatography on 60 Å silica gel (70-230 mesh) with a gradient solvent system. The gradient ran from 10% dichloromethane in hexane to 100% methanol. Following TLC evaluation, collection vials were combined to yield 10 distinct fractions (Table 2), which were evaluated for potential antidiabetic activity in the in vitro panel of assays.

TABLE 2
Silica gel column chromatography of the
ethyl acetate extract (D12101-D001)

| Fraction Code | Fraction number | Mass (g) |
|---|---|---|
| D12101-F002 | 1 | 0.0479 |
| D12101-F003 | 2 | 0.2143 |
| D12101-F004 | 3 | 2.64 |
| D12101-F005 | 4 | 9.14 |
| D12101-F006 | 5 | 7.04 |
| D12101-F007 | 6 | 10.03 |
| D12101-F008 | 7 | 44.3 |
| D12101-F009 | 8 | 0.7 |
| D12101-F0010 | 9 | 3.12 |
| D12101-F011 | 10 | 2.89 |

### NFκB Assay

To assess the ability of natural product extracts and compounds to inhibit the specific binding of activated NFκB transcription factor to the biotinylated consensus sequence, the EZ-Detect™ Transcription Factor Assay kit (Pierce Biotechnology) was used according to the kit manual and established protocol. HeLa cells (ATCC) were treated with each test sample and TNF-α, nuclear extracts harvested using the NE-PER kit (Pierce Biotechnology), and protein content equilibrated using the Pierce BCA Protein Assay kit (Pierce Biotechnology). Specific binding was detected through quantification of chemiluminescent signal in a plate reader (FLUOstar Optima, BMG Labtechnologies GmbH, Inc.). Nuclear extracts treated with TNF-α (Thermo Scientific) and rocaglamide (Enzo Life Sciences, Inc.) were used as negative and positive controls, respectively. Initial screens were performed with compounds and extracts at 50 μg/mL (Table 3), and data were reported as percent inhibitions of NFκB translocation, with the positive control rocaglamide normalized to 100%. Isolated compounds were investigated further by analyzing four concentrations (50, 5, 0.5, and 0.05 μg/mL) to determine the IC50 values by non-linear regression analysis using Table Curve 2D v4 (System Software Inc., San Jose, CA, USA).

TABLE 3
NFκB inhibitory activity of mace ethyl acetate
extract and fractions at 50 μg/mL

| Mace Sample | % inhibition NFκB |
|---|---|
| D12101-D001 | 95.3 |
| D12101-F002 | NT |
| D12101-F003 | 56.4 |
| D12101-F004 | <50 |
| D12101-F005 | 118.2 |
| D12101-F006 | <50 |
| D12101-F007 | 90.9 |
| D12101-F008 | <50 |
| D12101-F009 | <50 |
| D12101-F0010 | NT |
| D12101-F011 | NT |

### PARP-5 Enzyme Assay

Poly (ADP-ribose) polymerase 5 (PARP-5, also known as tankyrase-1 or TNKS1), is a nuclear enzyme involved in repairing single strand DNA breaks. Agonists of PARP-5 induce DNA repair, but the cost of PARP-5 activation is to slow glycolysis and limit GLUT-4-mediated glucose uptake. Small-molecule inhibition of the PARP-5 enzyme potentiates antidiabetic activities by increasing glucose uptake and insulin sensitivity. Extracts and compounds from the arils of M. fragrans were tested by using the TNKS1 Histone Ribosylation Assay kit (BPS Bioscience Inc.) according to the kit protocol. First, microplate wells were coated with immobilized histone substrate (50 μL) overnight at 4° C., then washed 3 times with PBS-Tween (200 μL). The wells were treated with blocking buffer and washed 3 times with PBS-Tween. The reaction mixture, which contained TNKS assay buffer and NAD+, was added to each sample well. Diluted sample solutions were added to the corresponding wells to achieve final concentrations of 50 μg/mL, followed by TNKS1 enzyme. After 1 hour, the plates were washed 3 times with PBS-Tween and incubated with primary antibody (1:800) for another hour. Following PBS-Tween wash, the secondary-HRP antibody was added (1:1000), incubated for 30 minutes, and finally chemiluminescent substrate was added. The plates were immediately measured for luminescence in the FLUOstar Optima plate reader (BMG Labtechnologies, GmbH, Inc.). The positive control included TNKS1 enzyme, NAD+, and DMSO; the substrate control included enzyme and DMSO; and the negative control included NAD+ and DMSO.297,304 The mace ethyl acetate fractions were screened for PARP-5 inhibition (Table 4).

TABLE 4
PARP-5 inhibitory activity of mace
ethyl acetate fractions at 50 μg/mL

| Mace Sample | PARP-5 % inhibition |
|---|---|
| D12101-F002 | NT |
| D12101-F003 | 92.95 |
| D12101-F004 | <50 |
| D12101-F005 | <50 |
| D12101-F006 | <50 |
| D12101-F007 | <50 |
| D12101-F008 | <50 |
| D12101-F009 | <50 |
| D12101-F0010 | NT |
| D12101-F011 | NT |

### PPAR-γ ELISA Assay

To evaluate the ability of natural product extracts and compounds to induce specific binding of PPAR-γ transcription factor in nuclear extracts to immunosorbed peroxisome proliferator response element, the PPAR-α,γ,δ Complete Transcription Factor Assay Kit (Cayman Chemical Company) was used according to the kit protocol. 303 CCD-112CoN normal colon cells or Hela cells (ATCC) were treated with mace extracts or isolated compounds for 24 h, nuclear extracts were harvested using the NE-PER kit (Pierce Biotechnology), and protein content equilibrated using the Pierce BCA Protein Assay kit (Pierce Biotechnology). Specific binding was detected through quantification of absorptivity at 630 nm in a BioTek uQuant microplate reader (BioTek Instruments, Inc.). Nuclear extracts treated with DMSO and rosiglitazone were used as negative and positive controls, respectively. Initial screens were performed with compounds and extracts at 50 μg/mL, and compounds investigated further were tested at four concentrations to determine their IC50 values by non-linear regression analysis. Ethyl acetate fractions F003 through F008 were screened for PPAR-γ agonism at 50 μg/mL (Table 5), reported as percent activation of PPAR-γ with positive control rosiglitazone normalized to 100%.

TABLE 5
PPAR-γ activation of mace ethyl acetate fractions at 50 μg/mL

| Mace Sample | PPAR-γ % inhibition |
|---|---|
| D12101-F002 | NT |
| D12101-F003 | 90.48 |
| D12101-F004 | 100 |
| D12101-F005 | 96.6 |
| D12101-F006 | 99.32 |
| D12101-F007 | 87.76 |
| D12101-F008 | 108.16 |
| D12101-F009 | NT |
| D12101-F0010 | NT |
| D12101-F011 | NT |

### Isolation of Licarin B (K033) and Licarin A (K034)

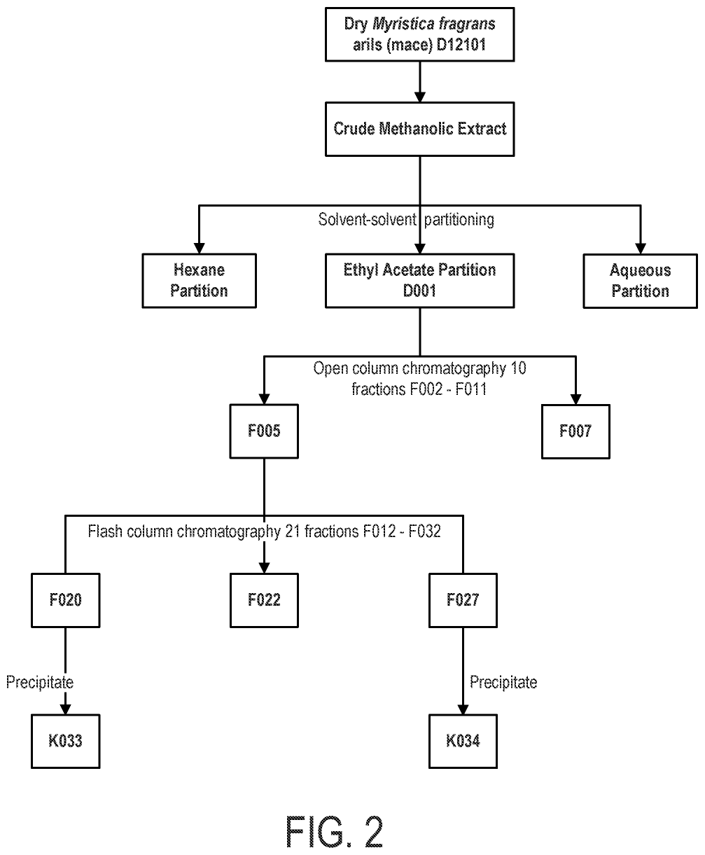

Due to its NFκB and PPAR-γ activity, fraction F005 was selected for further fractionation. Fraction F005 was subjected to flash column chromatography on slurry-packed 60 Å silica gel (230-400 mesh, 225 g). The sample (4.46 g) was adsorbed 2:3 onto gross silica gel (70-230 mesh) and dry-loaded to maintain an even sample bed. Gradient elution began with 5% ethyl acetate in hexane (2.4 L), followed by 10% (2.3 L), 20% (1.5 L), 50% (1.5 L), and 100% ethyl acetate (1 L). The column was washed with 50% ethyl acetate in methanol to retrieve the remaining sample, and 67 subfractions of approximately 125 ml were collected and combined according to TLC analysis (Table 6 and FIG. 2). Fractions F020 and F027 formed precipitates (K033 and K034, respectively), which were analyzed for purity by ₁H NMR spectroscopy. The precipitates were later identified as the purified compounds licarin B (K033, 41) and licarin A (K034, 42). Further fractionation was then performed on F022 since it contained the most material, appeared significantly different from K033 and K034 by TLC, and had well-separated composition profile on TLC.

TABLE 6
Flash silica gel column chromatography of D12101-F005

| Fraction Code | Fraction Number | Mass (g) |
|---|---|---|
| D12101-F012 | 1 | 0.0041 |
| D12101-F013 | 2 | 0.0070 |
| D12101-F014 | 3 | 0.0070 |
| D12101-F015 | 4 | 0.0030 |
| D12101-F016 | 5 | 0.0126 |
| D12101-F017 | 6-8 | 0.0488 |
| D12101-F018 | 9, 11 | 0.0823 |
| D12101-F019 | 10 | 0.1185 |
| D12101-F020 | 12-17 | 0.6973 |
| D12101-F021 | 18-21 | 0.0640 |
| D12101-F022 | 22-27 | 0.7923 |
| D12101-F023 | 28-33 | 0.1329 |
| D12101-F024 | 34-35 | 0.1117 |
| D12101-F025 | 36-39 | 0.1411 |
| D12101-F026 | 40-44, 47-49 | 0.1618 |
| D12101-F027 | 45 | 0.2030 |
| D12101-F028 | 46 | 0.1329 |
| D12101-F029 | 50-60 | 0.0989 |
| D12101-F030 | 61-62 | 0.0161 |
| D12101-F031 | 63-67 | 0.0104 |
| D12101-F032 | wash | 0.1152 |

### Isolation of Non-Polar Secondary Metabolite K040

Flash column chromatography was performed on 493.7 mg of fraction F022. The column was slurry-packed with 15 g fine (230-400 mesh) silica gel with the sample adsorbed onto 0.5 g gross (70-230 mesh, 60 Å) silica gel. Since F022 had a retention factor (R_{f}) of 0.3 by TLC in a 10% ethyl acetate in hexane mobile phase, the sample was eluted from the column utilizing a gradient solvent system from 10% to 100% ethyl acetate. Altogether, 45 fractions were collected and combined according to TLC analysis (Table 7). Fraction F040 was checked for purity by 1H NMR spectroscopy, but since the spectrum displayed signals diagnostic of a fatty acid in the 1-3 ppm range, structure elucidation was not pursued. Fractions F036 and F044 were selected for further purification due to their yields and apparent separation of components according to TLC.

TABLE 7
Flash silica gel chromatography of D12101-F022

| Fraction Code | Fraction number | Mass (g) |
|---|---|---|
| D12101-F035 | 1-3 | 0.065 |
| D12101-F036 | 4-5 | 0.208 |
| D12101-F037 | 6-11 | 0.005 |
| D12101-F038 | 12-13 | 0.002 |
| D12101-F039 | 14-20 | 0.006 |
| D12101-F040 | 21-29 | 0.002 |
| D12101-F041 | 30-31 | 0.001 |
| D12101-F042 | 32 | 0.005 |
| D12101-F043 | 33 | 0.001 |
| D12101-F044 | 34-40 | 0.004 |
| D12101-F045 | 41-45 | 0.007 |

### Reverse-Phase Chromatography of a Bilevel Combined Fraction

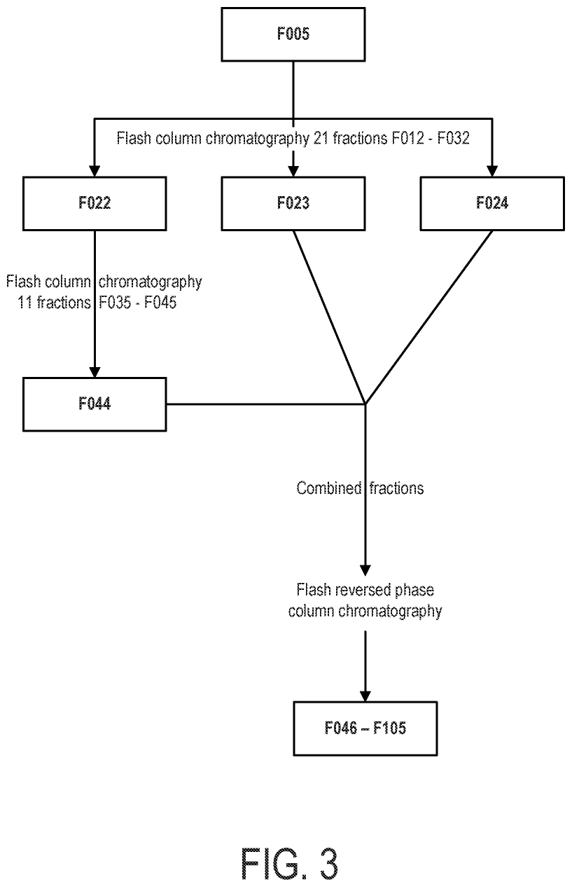

Fraction F044 exhibited a good separation profile on TLC and did not appear to be a very complex fraction. However, only 4 mg of the extract were obtained, so it was combined with two fractions from the previous column (F023 and F024) that had similar TLC separation profile. A reversed-phase C₁₈ column was used to separate the bilevel combined fractions (FIG. 3): F023 (18 mg), F024 (18 mg), and F044 (4 mg). With a gradient mobile phase from 50% water in methanol to 100% methanol, 680 fractions were collected through flash reversed-phase column chromatography and recombined according to fraction similarity by TLC (Table 8). Several of the fractions were evaluated by 1H NMR spectroscopy to determine purity, and upon finding no pure compounds, five fractions were selected for further separation by reversed-phase HPLC (F098-F102).

TABLE 8
Reversed-phase Cis column chromatography of
combined fractions D12101-F023, F024 and F044

| Fraction | Fraction | Fraction | Fraction | Mass |
|---|---|---|---|---|
| Code | number | Code | number | (g) |
| F046 | 1-3 | F076 | 270-279 | |
| F047 | 4-14 | F077 | 280-283 | |
| F048 | 15-54 | F078 | 284-290 | |
| F049 | 55-108 | F079 | 291-293 | |
| F050 | 85 | F080 | 294-299 | |
| F051 | 109-119 | F081 | 300-303 | |
| F052 | 120-130 | F082 | 304-308 | |
| F053 | 131-135 | F083 | 309-318 | |
| F054 | 136-150 | F084 | 319-320 | |
| F055 | 151-160 | F085 | 321-332 | 0.0014 |
| F056 | 161-167 | F086 | 333-336 | |
| F057 | 168-174 | F087 | 337-342 | |
| F058 | 175-181 | F088 | 344-356 | |
| F059 | 182-188 | F089 | 357-360 | 0.0003 |
| F060 | 189-195 | F090 | 361-364 | 0.0007 |
| F061 | 196-202 | F091 | 365-372 | |
| F062 | 203-209 | F092 | 373-384 | |
| F063 | 210-216 | F093 | 385-392 | |
| F064 | 217-219 | F094 | 393-396 | |
| F065 | 220-223 | F095 | 397-411 | |
| F066 | 224-227 | F096 | 412-420 | 0.0011 |
| F067 | 228-231 | F097 | 421-444 | 0.0027 |
| F068 | 232-234 | F098 | 445-460 | 0.0111 |
| F069 | 235-241 | F099 | 461-500 | 0.037 |
| F070 | 242-244 | F100 | 501-520 | 0.0126 |
| F071 | 245-246 | F101 | 521-540 | 0.011 |
| F072 | 247-254 | F102 | 541-580 | 0.0135 |
| F073 | 255-260 | F103 | 581-616 | |
| F074 | 261-267 | F104 | 617-640 | |
| F075 | 268-269 | F105 | 641-680 | |

Isolation of erythron-8.4′-Oxyneolign-8′-ene (K113) and Maceneolignan J (K126)

From a bilevel reversed-phase column, five fractions were separated individually by HPLC on a SunFire Prep RP18 column with a 7:3 methanol to water mobile phase (or 65:35 for F098). Fractions F098 and F099 eluted as two or five fractions that were collected, respectively, with retention times recorded in Table 9. By 1H NMR spectroscopy, it was discovered that peak 1 from F098 and peak 3 from F099 were the same compound, and the two were combined to yield 4.1 mg of compound K113. This was later identified as the known compound, erythro-8,4′-oxyneolign-8′-ene (43).

Additionally, four subfractions were collected from each of the F100, F101 and F102 HPLC separations (Table 9). Subfraction 4 from both F101 and F102 were combined due to their identical retention times and analyzed for purity by 1H NMR spectroscopy. The combined fraction appeared to be a semi-pure compound, K126, and its major isomer was later identified as the known compound maceneolignan J (44). All four HPLC fractions from F100 yielded less than 0.5 mg each and were not further investigated (with their retention times recorded in Table 9).

TABLE 9
Reversed-phase HPLC of D12101-F098 through D12101-F102

| | Collected | Retention | |
|---|---|---|---|
| Fraction Code | Subfraction | Time (min) | Compound |
| D12101-F098 | 1 (F106) | 34.9 | K113 (43) |
| | 2 (F107) | 37.5 | ND |
| D12101-F099 | 1 (F108) | 17.5 | ND |
| | 2 (F109) | 18.5 | ND |
| | 3 (F110) | 23.7 | K113 (43) |
| | 4 (F111) | 26.8 | ND |
| | 5 (F112) | 36.6 | ND |
| D12101-F100 | 1 (F114) | 17.5 | ND |
| | 2 (F115) | 20.2 | ND |
| | 3 (F116) | 23.7 | ND |
| | 4 (F117) | 26.8 | ND |
| D12101-F101 | 1 (F118) | 20.6 | ND |
| | 2 (F119) | 24.1 | ND |
| | 3 (F120) | 28.6 | ND |
| | 4 (F121) | 32.6 | K126 (44) |
| D12101-F102 | 1 (F122) | 20.7 | ND |
| | 2 (F123) | 24.0 | ND |
| | 3 (F124) | 28.4 | ND |
| | 4 (F125) | 32.6 | K126 (44) |

Characterization of Compounds Isolated from Myristica fragrans Arils

The structures of all natural products isolated from the arils of M. fragrans in the present investigation are shown below, with isolation yields presented in Table 10. The characterization of each of these four compounds will be described individually. The purities of three of the isolates (41-43) were verified by using TLC with three different solvent systems and ¹H NMR spectroscopy, or HPLC and 4H NMR spectroscopy. The fourth compound appeared pure by HPLC, but after ¹H NMR analysis, combined fractions F121 and F125 were deemed to be a mixture of two isomers (K126 and K127). The more abundant isomer, K126, was identified as 44.

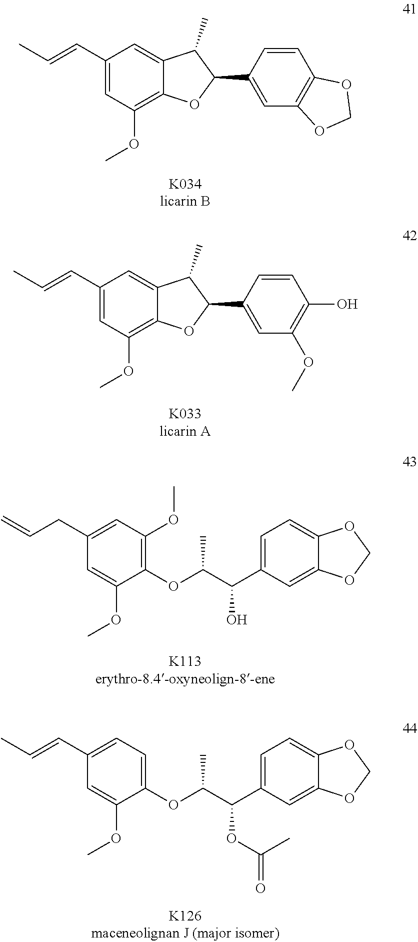 [SMILES: C/C=C/c1cc(OC)c2c(c1)[C@H](C)[C@@H](c1ccc3c(c1)OCO3)O2 ; C/C=C/c1cc(OC)c2c(c1)[C@H](C)[C@@H](c1ccc(O)c(OC)c1)O2 ; C=CCc1cc(OC)c(O[C@H](C)[C@@H](O)c2ccc3c(c2)OCO3)c(OC)c1 ; C/C=C/c1ccc(O[C@H](C)[C@@H](OC(C)=O)c2ccc3c(c2)OCO3)c(OC)c1]

TABLE 10
Isolation yields of compounds isolated
from M. fragrans in present example

| Compound Code | Structure | Yield (mg) |
|---|---|---|
| D12101-K034 | 41 | 29.7 |
| D12101-K033 | 42 | 125.8 |
| D12101-K113 | 43 | 4.1 |
| D12101-K126 | 44 | 1.2 |

### Characterization of K034, Licarin B (41)

White amorphous solid; [α]²⁰_{D}+30.0 (c 0.1, MeOH), ¹H NMR: see Table 11, BC NMR: see Table 12, HRESIMS observed m/z 325.14337 [M+H]⁺ (calculated for C₂₀H₂₀O₄H, 325.143436).

TABLE 11
1H NMR data for mace isolates 41-44, reported at 8H (ppm)

| | Compound: | | | |
|---|---|---|---|---|
| | 41 | 42 | 43 | 44 |
| | Proton | | | |
| | (CDCl₃) | (CDCl₃) | (CD₃OD) | (CD₃OD) |
| 2 | 6.93, d 1.6 | 6.98, s | 6.74, s | 6.94, d 2.0 |
| 5 | 6.78, d 8.0 | 6.90, m | 6.86, m | 6.77, d 8.0 |
| 6 | 6.88, dd, 8.0, | 6.91, m | 6, 74, s | 6.83, dd 8.0, 1.6 |
| | 2.0 | | | |
| 7 | 5.10, d 8.8 | 5.11, d 9.6 | 4.73 d 3.6 | 5.78, d 4.0 |
| 8 | 3.41, dq 7.6, 7.6 | 3.45 dq 9.2, 6.8 | 4.26 dq 6.4, 3.6 | 4.56 d 4.4 |
| 9 | 1.38, d 6.8 | 1.39, d 6.8 | 1.09, d 6.4 | 1.23, d 6.4 |
| 2′ | 6.75, s | 6.77, s | 6.53, s | 6.83, d 1.6 |
| 3′ | | | | 6.90, d 8.4 |
| 6′ | 6.78, s | 6.80, s | 6.53, s | 6.96, d 1.2 |
| 7′ | 6.36, dd 15.6, | 6.37, dd 15.6, | 3.35, dt 6.8, 1.6 | 6.33, dd 15.6, |
| | 1.6 | 1.6 | | 1.6 |
| 8′ | 6.10, dq 15.6, | 6.11, dq 15.6, | 5.98, qt 10.-, 6.8 | 6.15, dq 15.6, |
| | 6.8 | 6.8 | | 6.4 |
| 9′ | 1.86, dd 6.8, 1.6 | 1.88, dd 6.8, 1.6 | | 1.84, dd 6.4, 1.6 |
| 9a′ | | | 5.06, ddt 10.0, | |
| | | | 2.0, 1.2 | |
| 9b′ | | | 5.11, ddt 16.8, | |
| | | | 2.0, 1.6 | |
| 3-OMe | | 3.88, s | | |
| 4-OH | | 5.67, s | | |
| 3′-OMe | | | 3.83, s | |
| 5′-OMe | 3.89, s | 3.90, s | 3.83, s | 3.81, s |
| O—CH₂—O | 5.95, s | | 5.90, t | 5.92, s |
| OAc (CH₃) | | | | 2.08, s |

TABLE 12
¹³C NMR Data for mace isolates 41-44, reported as δ_{C} (ppm)

Compound:

| | | | |
|---|---|---|---|
| 41 | 42 | 43 | 44 |

Carbon

| | (CDCl₃) | (CDCl₃) | (CD₃OD) | (CD₃OD) |
|---|---|---|---|---|
| 1 | 134.4 | 132.4 | 136.2 | 134.6 |
| 2 | 106.8 | 109.0 | 108.7 | 108.9 |
| 3 | 147.9 | 146.7 | 149.0 | 149.1 |
| 4 | 147.6 | 145.8 | 148.1 | 147.4 |
| 5 | 108.1 | 114.1 | 108.0 | 108.8 |
| 6 | 120.2 | 120.0 | 120.8 | 122.1 |
| 7 | 93.4 | 93.8 | 75.6 | 78.3 |
| 8 | 45.8 | 45.6 | 83.6 | 79.4 |
| 9 | 17.9 | 17.6 | 13.8 | 15.6 |
| 1′ | 132.3 | 132.1 | 137.8 | 132.5 |
| 2′ | 113.4 | 113.4 | 106.9 | 119.5 |
| 3′ | 133.1 | 133.3 | 154.8 | 119.9 |
| 4′ | 146.5 | 146.6 | 134.7 | 148.9 |
| 5′ | 144.1 | 144.4 | 154.8 | 152.4 |
| 6′ | 109.4 | 109.3 | 106.9 | 111.3 |
| 7′ | 131.0 | 131.0 | 41.5 | 131.9 |
| 8′ | 123.5 | 123.5 | 138.8 | 125.2 |
| 9′ | 18.4 | 18.4 | 116.2 | 18.5 |
| 3-OMe | — | 56.0 | — | — |
| 3′-OMe | — | — | 56.6 | — |
| 5′-OMe | 56.0 | 56.0 | 56.6 | 56.5 |
| O—CH₂—O | 101.1 | — | 102.2 | 102.5 |
| OAc (CH₃) | — | — | — | 21.0 |
| OAc (CO₂) | — | — | — | 171.9 |

### Characterization of K033, Licarin A (42)

White powder; [α]²⁰_{D}+4.0 (c 0.1, MeOH) ¹H NMR: see Table 11; ¹³C NMR: see Table 12; HRESIMS observed m/z 327.15899 [M+H]⁺ (calculated for C₂₀H₂₂O₄H, 327.159086).

Characterization of K113, erythron-8.4′-oxyneolign-8′-ene (43)

White amorphous solid; [α]²⁰_{D}+34.0 (c 0.05, MeOH) ¹H NMR: see Table 11; ¹³C NMR: see Table 12; HRESIMS observed m/z 395.14640 [M+Na]⁺ (calculated for C₂₁H₂₄O₆Na, 395.146509).

### Characterization of K126, Maceneolignan J (44)

Colorless oil; [α]²⁰_{D}+73.3 (c 0.075, MeOH)₁H NMR: see Table 11; ¹³C NMR: see Table 12; HRESIMS observed m/z 407.14641 [M+Na]⁺ (calculated C₂₂H₂₄O₆Na, 407.146509).

Bioactivity of Isolated Compounds 41-44 from Mace

The four compounds isolated from mace (41-44) were tested in the panel of in vitro assays to determine their potential antidiabetic activity. The isolates were evaluated in the NFκB assay at 50, 5, 0.5, and 0.05 μg/mL, according to the methods detailed herein, to determine IC₅₀ values (Table 13). The isolates were also screened in the PPAR-γ (Table 14) and PARP-5 assays (Table 15) at concentrations of 50 μg/mL, according to the experimental methods herein.

TABLE 13
NFKB activity of isolated 41-44

| Compound | NFκB IC₅₀ | |
|---|---|---|
| 41 | 474.8 | nM |
| 42 | 30.6 | nM |
| 43 | 64.4 | nM |
| 44 | 0.1 | nm |

TABLE 14
PPAR-γ activity of isolates 41-44 at 50 μg/mL

| Compound | PPAR-γ % activation |
|---|---|
| 41 | 45 |
| 42 | 81 |
| 43 | <50 |
| 44 | NT |

TABLE 15
PARP-5 activity of isolates 41-44 at 50 μg/mL

| Compound | PARP-5% inhibition |
|---|---|
| 41 | <50 |
| 42 | <50 |
| 43 | NT |
| 44 | NT |

### Derivatization of K033, Licarin A (42)

To improve potential antidiabetic properties of licarin A for the purpose of drug discovery and development and to probe possible mechanisms of action, 42 was derivatized to form compounds 45, 46, 47, and 48.

### Derivatization Reactions

### Etherification

A scintillation vial was charged with licarin A (42) (45 mg, 0.1379 mmol, 1 equiv.), followed by potassium carbonate (95.28 mg, 0.4137 mmol, 3 equiv.) and acetone (1 mL). 2-Bromopropane (25.89 UL, 0.2758 mmol, 2 equiv.) was then charged to the vial and heated to 70° C. for 12 h. After cooling to room temperature, the reaction mixture was filtered through a syringe filter (0.45 μm), concentrated to dryness, and purified via reversed-phase HPLC. After purification and characterization, it was determined that the etherification occurred, along with an unexpected oxidative cleavage of the olefin to the aldehyde.

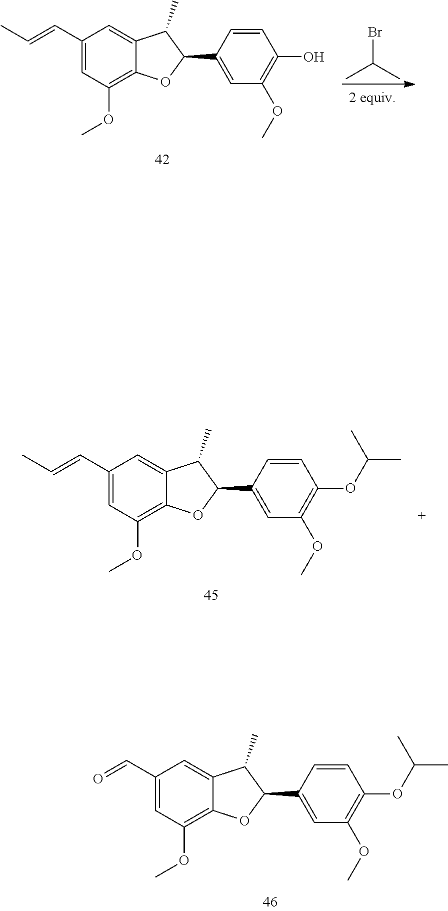

### Esterification

A scintillation vial was charged with licarin A (42) (45 mg, 0.1379 mmol, 1 equiv.), followed by potassium carbonate (95.28 mg, 0.4137 mmol, 3 equiv.) and acetone (1 mL). Ethyl chloroformate (26.36 μL, 0.2758 mmol, 2 equiv.) was then charged to the vial and heated to 70° C. for 12 h. After cooling to room temperature, the reaction mixture was filtered through a syringe filter (0.45 μm), concentrated to dryness, and purified via reversed-phase HPLC. After purification and characterization, it was determined that the esterification occurred, along with an unexpected oxidative cleavage of the olefin to the aldehyde.

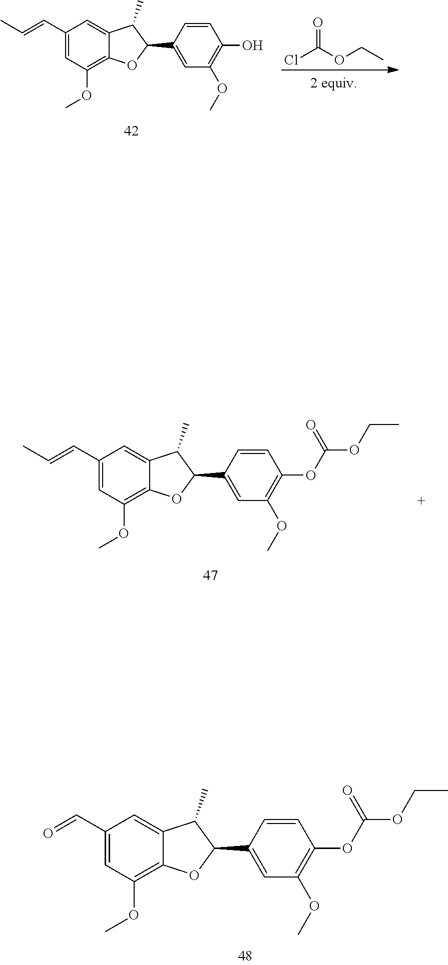

### Oxidative Cleavage

A sodium periodate-mediated oxidative cleavage was performed in a scintillation vial charged with licarin A (42) (20 mg, 0.0613 mmol, 1 equiv.), sodium metaperiodate (32.77 mg, 0.1532 mmol, 2.5 equiv.), and a catalytic amount of osmium tetroxide. Water (500 μL), ethanol (500 L), tetrahydrofuran (100 μL), and a catalytic amount of sulfuric acid were then charged to the vial and heated to 70° C. for 12 h. After cooling to room temperature, the reaction mixture was subjected to liquid-liquid extraction with chloroform (3 mL, 3 times). The chloroform extract was then filtered through a syringe filter (0.45 μm), concentrated to dryness, and purified via reversed-phase HPLC.

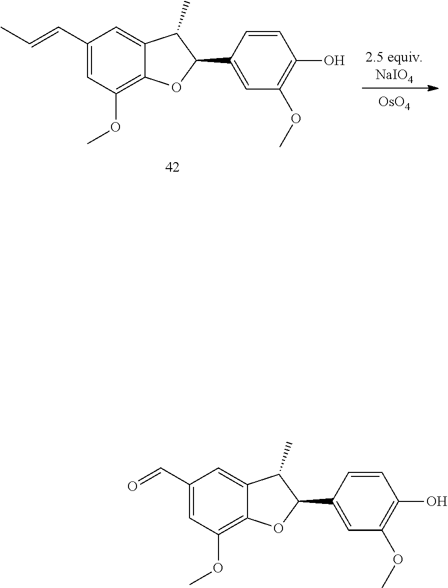

### Purification of Licarin Derivatives

### Separation of Etherification Reaction Products

Products from the etherification reaction for derivatizing licarin A (42) were separated by reversed-phase HPLC using the Waters SunFire column with the isocratic mobile phase 7:3 methanol and water for 8 minutes. Serial injections of approximately 6 mg/mL of the reaction products were separated and two peaks collected at initial retention times 3.1 and 4.2 mins. Peak 1 (Rt=3.1 min) was later identified as licarin A starting material (42), and the second peak was checked for purity by ¹H NMR spectroscopy.

Since ¹H NMR spectroscopy indicated that peak 2 contained a mixture of several highly similar compounds, further purification was performed via reversed-phase HPLC. A longer column was selected, given the expected similarity of the reaction products. A Varian Polaris C₁₈-A column was utilized with an isocratic mobile phase of 1:1 methanol and water. Serial injections of 50 μL at approximately 9 mg/mL were separated over the course of 15 minutes into five fractions. The two peaks with the highest absorption and largest integration area had initial retention times of 11.1 and 12.7 min and were later identified as 46 and 45, respectively. Both fractions were evaluated for purity by ¹H NMR spectroscopy, followed by acquisition of their ¹³C NMR and HMBC spectra. Yields of purified derivatives from this etherification and the esterification reactions are presented in Table 16.

TABLE 16
Derivative yields

| | Initial Retention | Peak Retention | Yield |
|---|---|---|---|
| Compound | Time (min) | Time (min) | (mg) |
| 42 | 2.8 | 3.1 | ND |
| Mixture | 3.8 | 4.2 | 19.45 |
| 46 | 11.1 | 11.2 | 4.18 |
| 45 | 12.7 | 13.1 | 3.81 |
| 48 | 10.4 | 10.9 | 2.51 |
| 47 | 11.9 | 12.2 | 3.44 |

### Separation of Esterification Reaction Products

The reaction mixture from the esterification derivatization reaction was separated by reversed-phase HPLC using the Agilent Varian Polaris C18-A column. The reaction mixture was injected serially at 25 mg/mL with injection volumes of up to 100 μL. An isocratic mobile phase of 1:1 acetonitrile and water eluted five significant peaks over the course of 15 mins. All five peaks were collected as individual fractions and evaluated for purity by ¹H NMR spectroscopy. Two fractions appeared to be pure compounds (later identified as 47 and 48), and thus their ¹H, ¹³C, and HMBC spectra were obtained. The yields of 47 and 48 are presented in Table 16.

### Separation of Oxidative Cleavage Reaction Products

The reaction mixture from the oxidative cleavage derivatization reaction was separated by reversed-phase HPLC on the Varian column. Despite considerable optimization efforts using various mobile phase conditions including methanol, acetonitrile, water, and acetic acid, no distinct separation could be achieved. The best mobile phase was 1:1 acetonitrile and water, run isocratically for 15 minutes. Since monitoring peak separation at different absorption wavelengths was far from ideal, eluted fractions were collected based almost entirely on retention time. Five fractions with initial retention times of 9.6, 10.4, 11.4, 11.9, and 12.7 mins, corresponding to the most distinct peak shoulders, were collected and evaluated for purity by ¹H NMR spectroscopy. As expected according to the unimpressive HPLC trace, none of the collected fractions were pure and isolation attempts were abandoned, as a consequence to having previously acquired two different aldehyde products (46 and 48).

### Characterization of Licarin A Derivatives

Characterization was completed by analysis of 1D- and 2D-NMR and MS data. Reference is made in view of the numbering systems of 8,3′-type neolignans and 8-O-4′-type neolignans as provided below.

### Number System for 8,3′-Type Neolignans

| | R₁ | R₂ | R₃ | |
|---|---|---|---|---|
| 41 | —CHCH₃ | O—CH₂—O | | |
| 42 45 46 | —CHCH₃ —CHCH₃ O | OH OCH(CH₃)₂ OCH(CH₃)₂ | OMe OMe OMe | 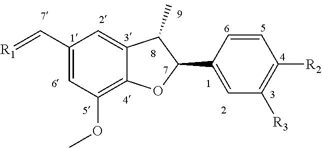 |
| 47 | —CHCH₃ | 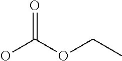 | OMe | |
| 48 | O | 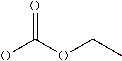 | OMe | |

### Number System for 8-O-4′-Type Neolignans

| 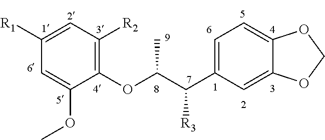 | | | |
|---|---|---|---|
| | R₁ | R₂ | R₃ |
| 43 | —CH₂HC═CH₂ | OMe | OH |
| 44 | —CH₂HC═CH | H | OAc |

### Characterization of Isopropxy Licarin A (45)

White amorphous solid; [α]²⁰_{D}+9.0 (e 0.1, MeOH); ¹H NMR: see Table 17; BC NMR: see Table 18, HRESIMS observed m=369.20593 [M+H]⁺ (calculated for C₂₈H₂₈O₄H, 369.206036).

TABLE 17
¹H NMR data comparing derivatives 45-48 to licarin A (42), reported as δ_{H} (ppm)

| | Compound: | | | | |
|---|---|---|---|---|---|
| | 42 | 45 | 46 | 47 | 48 |
| | Proton: | | | | |
| | CDCl₃ | CDCl₃ | CDCl₃ | CDCl₃ | CDCl₃ |
| 2 | 6.98, s | 6.98, d 2.0 | 6.95, d 2.0 | 7.07, d 1.6 | 7.04, d 2.0 |
| 5 | 6.90, m | 6.86, d 8.0 | 6.88, d 8.0 | 7.11, d 8.4 | 7.14, d 8.4 |
| 6 | 6.91, m | 6.92, dd 8.4, | 6.93, dd ND | 6.97, dd 8.4, | 6.98, dd 8.0, |
| | | 2.0 | | 1.6 | 1.6 |
| 7 | 5.11, d 9.6 | 5.11, d 9.6 | 5.26, d 9.2 | 5.16, d 8.8 | 5.31, d 9.2 |
| 8 | 3.45, dq 9.2, | 3.48, dq 6.8, | 3.58, dq 6.8, | 3.45, dq 7.2, | 3.56, dq 7.6, |
| | 6.8 | 6.4 | 6.4 | 6.8 | 7.2 |
| 9 | 1.39, d 6.8 | 1.39, d 6.8 | 1.46, d 6.4 | 1.41, d 6.8 | 1.48, d 6.8 |
| 2′ | 6.77, s | 6.77, s | 7.38, d 1.2 | 6.76, s | 7.39, d 1.2 |
| 6′ | 6.80, s | 6.78, s | 7.35, t 1.2 | 6.79, s | 7.34, t 1.2 |
| 7′ | 6.37, dd | 6.36, dd | 9.85, s | 6.36, dd | 9.86, s |
| | 15.6, 1.6 | 15.6, 1.6 | | 15.6, 1.6 | |
| 8′ | 6.11, dq | 6.11, dq | | 6.11, dq | |
| | 15.6, 6.8 | 15.6, 6.4 | | 15.6, 6.8 | |
| 9′ | 1.88, dd 6.8, | 1.87, dd 6.4, | | 1.87, dd 6.8, | |
| | 1.6 | 1.6 | | 1.6 | |
| 3-OMe | 3.88, s | 3.84, s | 3.85, s | 3.85, s | 3.86, s |
| 4-OH | 5.67, s | | | | |
| 5′-OMe | 3.90, s | 3.89, s | 3.95, s | 3.90, s | 3.96, s |
| 3-OIPr | | 1.36, d 6.0 | 1.37, d 6.8 | | |
| (CH₃)₂ | | | | | |
| 4-OiPr (CH) | | 4.52, septet | 4.53, septet | | |
| | | 6.0 | 6.0 | | |
| 4-OEt (CH₃) | | | | 1.38, t 7.2 | 1.39, t 6.8 |
| 4-OEt (CH₂) | | | | 4.31, q 7.2 | 4.32, q 7.2 |

TABLE 18
¹³C NMR data comparing derivatives 45-48
to licarin A (42), reported as δ_{C} (ppm)

Compound:

| | | | | |
|---|---|---|---|---|
| 42 | 45 | 46 | 47 | 48 |

Carbon:

| | CDCl₃ | CDCl₃ | CDCl₃ | CDCl₃ | CDCl₃ |
|---|---|---|---|---|---|
| 1 | 132.4 | 133.1 | 132.0 | 140.0 | 138.5 |
| 2 | 109.0 | 110.4 | 110.4 | 110.4 | 110.4 |
| 3 | 146.7 | 150.5 | 150.6 | 151.4 | 151.5 |
| 4 | 145.8 | 147.5 | 147.9 | 139.5 | 140.3 |
| 5 | 114.1 | 115.5 | 115.6 | 122.3 | 122.6 |
| 6 | 120.0 | 119.2 | 119.3 | 118.7 | 118.6 |
| 7 | 93.8 | 93.7 | 94.9 | 93.1 | 94.2 |
| 8 | 45.6 | 45.5 | 44.7 | 45.9 | 45.1 |
| 9 | 17.6 | 17.7 | 17.9 | 17.9 | 18.1 |
| 1′ | 132.1 | 132.2 | 120.1 | 132.5 | 120.1 |
| 2′ | 113.4 | 113.3 | 110.4 | 113.4 | 111.9 |
| 3′ | 133.3 | 133.3 | 133.7 | 133.0 | 133.4 |
| 4′ | 146.6 | 146.6 | 153.3 | 146.5 | 153.2 |
| 5′ | 144.4 | 144.2 | 145.0 | 144.2 | 145.0 |
| 6′ | 109.3 | 109.3 | 111.8 | 109.4 | 111.9 |
| 7′ | 131.0 | 131.0 | 190.6 | 130.9 | 190.6 |
| 8′ | 123.5 | 12.5 | — | 123.7 | — |
| 9′ | 18.4 | 18.4 | — | 18.4 | — |
| 3-OMe | 56.0 | 56.0 | 56.1 | 56.0 | 56.1 |
| 5′-OMe | 56.0 | 56.0 | 56.1 | 56.1 | 56.1 |
| 4-OiPr | — | 22.1 | 22.0 | — | — |
| (CH₃) | | | | | |
| 4-OiPr | — | 22.1 | 22.1 | — | — |
| (CH₃) | | | | | |
| 4-OiPr | — | 71.5 | 71.5 | — | — |
| (CH) | | | | | |
| CO₂Et | — | — | — | 14.2 | 14.2 |
| (CH₃) | | | | | |
| CO₂Et | — | — | — | 65.0 | 65.0 |
| (CH₂) | | | | | |
| CO₂Et | — | — | — | 153.2 | 153.1 |

### Characterization of Isopropoxy Licarin A Carbaldehyde (46)

White amorphous solid; [α]²⁰_{D}+9.0 (c 0.1, MeOH); ¹H NMR: see Table 17; 1C NMR: see Table 18; HRESIMS observed m/z 357.16958 [M+H]⁺ (calculated for C₂₁H₂₄O₅H, 357.169650).

### Characterization of Licarin A Ethyl Carbonate (47)

White amorphous solid; [α]²⁰_{D}+35.0 (c 0.1, MeOH); ¹H NMR: see Table 17; 13C NMR: see Table 18; HRESIMS observed m/z 399.18007 [M+H]⁺ (calculated for C₂₃H₂₆O₆H, 399.180215).

### Characterization of Licarin A Formyl Ethyl Carbonate

White amorphous solid; [α]²⁰_{D}+21.0 (c 0.1, MeOH); ¹H NMR: see Table 17; 13C NMR: see Table 18; HRESIMS observed m/z 387.14374 [M+H]⁺ (calculated for C₂₁H₂₂₀₇H, 387.143829).

### Bioactivity of Licarin A Derivatives 45-48

Licarin A derivatives 45-48 were screened through the panel of in vitro assays to evaluate their potential as antidiabetic compound leads. PPAR-γ activities of the derivatives were tested at 50 μg/mL in Hela cells (Table 19). Methods for the assay were as described above.

TABLE 19
PPAR-γ activity of licarin A derivatives at 50 μg/mL

| Compound | PPAR-γ % activation |
|---|---|
| 45 | 57 |
| 46 | 29 |
| 47 | 0 |
| 48 | 0 |

### Evaluation of Mace Isolates and Derivatives in Diabetic Zebrafish

Following isolation, derivatization, and analysis of compounds 41-48 through the in vitro panel of assays, the mace natural products and licarin A derivatives were investigated in the larval diabetic zebrafish assay for potential antidiabetic activity. Western blot analysis of zebrafish expression levels for glycated hemoglobin, metabolic enzymes, and molecular targets NFκB and PPAR-γ was performed to evaluate potential lead improvement and to discover possible mechanisms of action.

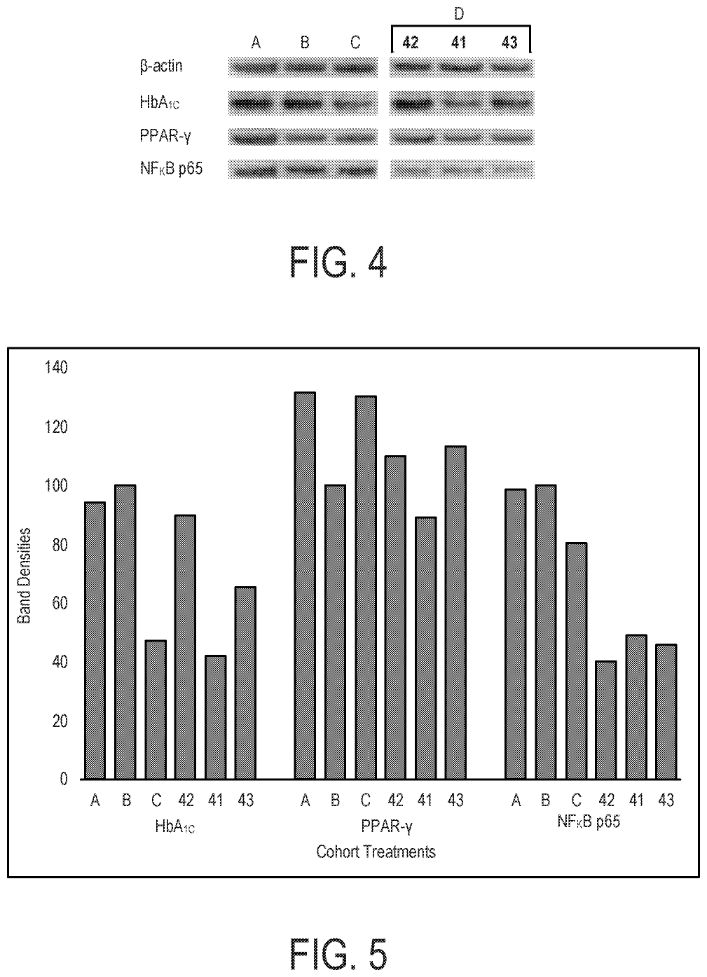

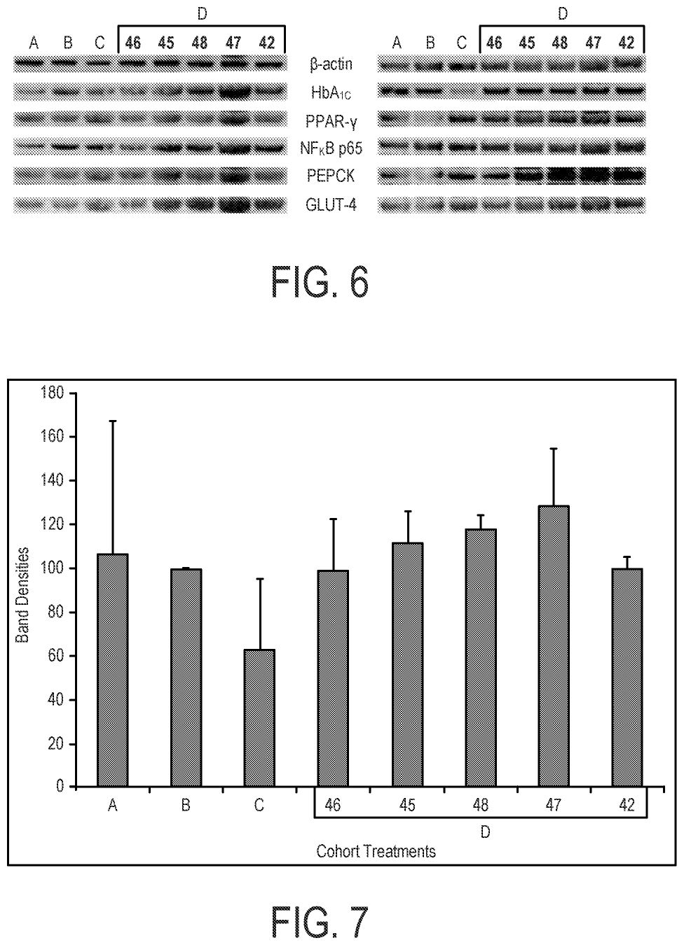

Wild-type zebrafish were sorted into 24-well microplates with 10 fish per well for a four-phase treatment cycle. At 72 hpf, type 2 diabetes was induced by administering 0.5 mg/mL alloxan in fish water for percutaneous absorption. After three hours of alloxan treatment, phase two of the treatment cycle was initiated by administering 10 μM doses of natural product compounds 41-43 (FIG. 4) or derivatives 45-48 (FIG. 6) with 0.01% DMSO in system water. At 78 hpf, the incubation solution was changed to 10 mg/ml D-glucose for 30 minutes, followed by a phase-four rinse in fresh system w Following the four-phase treatment cycle, zebrafish treatment groups were snap frozen in microcentrifuge tubes on dry ice, stored overnight at −80° C., thawed, and homogenized with 2 μL PBS per animal. Following centrifugation (60 minutes at 4° C. and 17,000 rpm), protein content of the homogenate supernatant was determined using the Pierce BCA Protein Assay kit (Pierce Biotechnology). Homogenates were analyzed by Western blot with equal amounts of total protein (20 μg) in each sample and using 4×LDS loading buffer (NuPAGE Life Technologies). The immunoblot samples were heated at 95° C. for 5 minutes and loaded onto NuPAGE Bis-Tris 4-12% gradient SDS-PAGE gels.

Electrophoresis was performed with NuPAGE MES SDS Running Buffer and using the XCell SureLock Mini-Cell Electrophoresis System (Life Technologies). Proteins separated on the gel were transferred to a PVDF membrane using TBST buffer. The blots were blocked in 3% BSA in TBST and subsequently probed overnight with primary antibodies (1:1000 in 1% BSA in TBST) against each target of interest. Following TBST rinses, secondary HRP-conjugated antibodies (1:2000) were applied to the membrane for 60 minutes. The HRP conjugated antibodies were detected using SuperSignal West Femto chemiluminescent substrate (Pierce Biotechnology) and immunoblot images were captured using the luminometer and Lumi Analyst 3.0 software.

Band densities were determined using ImageJ Gel Analysis to determine expression levels of each protein of interest. Expression levels of HbA_{1C}, PPAR-γ, and NFκB were organized into a bar graphs (FIG. 5) with band densities calculated in comparison to the housekeeping gene β-actin, normalized to the untreated diabetic group (B) of zebrafish. after for 60 minutes.

### Results and Discussion

### Antidiabetic Activity-Guided Fractionation of Mace

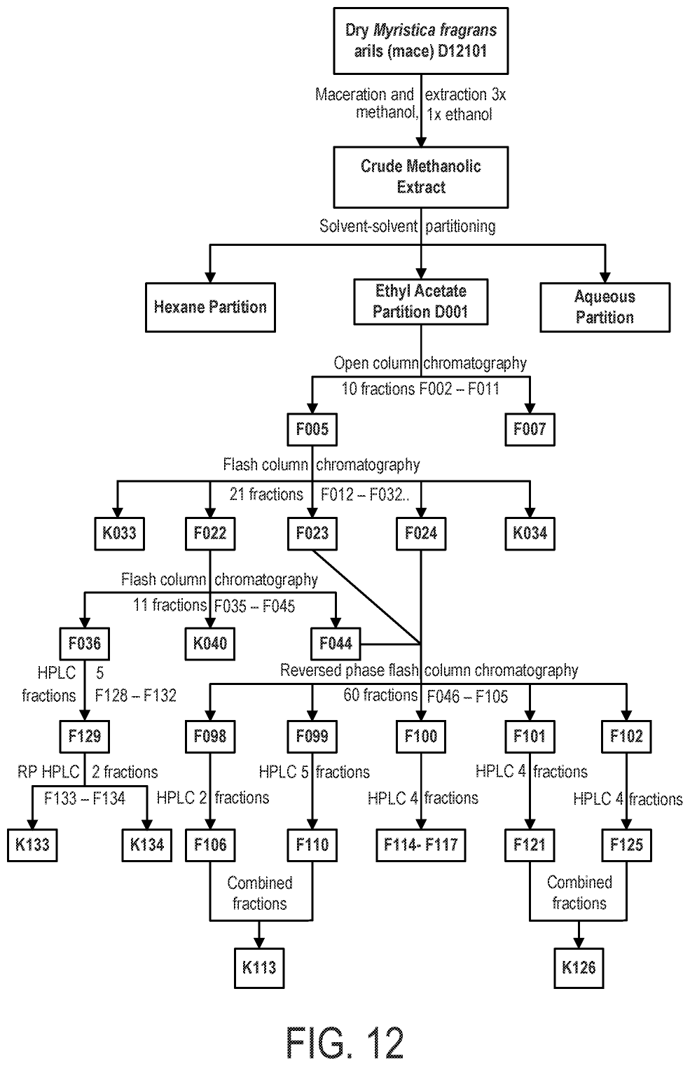

Through bioactivity-guided fractionation, four compounds were isolated from mace, their structures identified, and were determined as known natural products (see FIG. 12). In the extraction scheme (FIG. 12), the rectangles represent compounds that were isolated and exhibited ¹H NMR signals characteristic of fatty acids; as such, they did not fit the scope of this antidiabetic drug discovery project and were not elucidated or identified.

Bioactivity-guided fractionation that yielded the isolated and identified compounds began with the ethyl acetate partition (D12101-D001), which was active in the NFκB in vitro assay (95.3% as active as the positive control, rocaglamide, as reported in Table 3). In addition, based on polarity, it was expected to contain lignans, some of which have been reported previously to be PPAR agonists. 77,327,348 Therefore, the ethyl acetate partition was selected for further fractionation, which yielded ten fractions. Fraction D12101-F005 exhibited greater NFκB inhibition than rocaglamide (Table 3) and was 96.6% as active as the PPAR-γ positive control, rosiglitazone (Table 5). Fraction F005 was then separated by column chromatography, yielding two pure compounds, licarin B (41 or K033) and licarin A (42 or K034). Recombination of fractions, followed by separation by preparatory reversed-phase HPLC yielded D12101-K113 and D12101-K126, identified in the following section as 43 and 44, respectively.

Biological Evaluation of the Compounds Isolated and Derivatized from M. Fragrans Bioactivity of Mace Isolates and Derivatives in the in vitro Panel

The isolated compounds (41-44) and licarin A derivatives (45-48) identified and elucidated were evaluated for potential antidiabetic activity through a panel of in vitro assays. To summarize the assay panel results have been combined for easy reference in Table 20.

TABLE 20
Summary of results for panel of in vitro assays to evaluate potential
antidiabetic activity of mace isolates 41-44 and derivatives 45-48

| | | | PPAR-γ % | PARP-5% |
|---|---|---|---|---|
| Compound | NFκBIC₅₀ | | activation | inhibition |
| 41 | 478.8 | nM | 45 | <50 |
| 42 | 30.6 | nM | 81 | <50 |
| 43 | 64.4 | nM | <50 | NT |
| 44 | 0.1 | nM | NT | NT |
| 45 | NT | | 57 | NT |
| 46 | NT | | 29 | NT |
| 47 | NT | | 0 | NT |
| 48 | NT | | 0 | NT |

The fraction that yielded isolates 41-44 (D12101-F005) was not active in the PARP-assay (Table 4) but was 96.6% as effective as the positive control rosiglitazone (in the preliminary evaluation of extracts tested at 50 μg/mL, Table 5) in acting as an agonist on PPAR-γ in the in vitro assay. Generally, the compounds isolated from an extract by bioassay-guided fractionation have a greater probability of demonstrating PPAR-γ activity than compounds isolated without consideration of the parent fraction biological activity. As shown in Table 20, the first two compounds isolated from D12101-F005 (licarin B, 41 and licarin A, 42) were not effective PARP-5 inhibitors, which was in alignment with the parent fraction, and the compounds isolated later (43 and 44) and the licarin A derivatives (45-48) were not tested in the assay. On the other hand, licarin A (42) exhibited moderate PPAR-Y activity in vitro. Unfortunately, the other natural product compounds and the licarin A derivatives had little to no PPAR-γ activity in the preliminary screening procedure. Maceneolignan J (44) was not evaluated in the PPAR-γ in vitro assay due to its low isolation yield and partial contamination with a similar isomer.

Since fraction D12101-F005 had excellent NFκB inhibitory activity (118% as active as positive control rocaglamide in the preliminary evaluation of extracts tested at 50 μg/mL, Table 3), its components also had great potential to be identified as compounds that could inhibit NFκB translocation and the subsequent inflammatory reaction. As shown in Table 20, each of the natural product compounds exhibited nanomolar half maximal inhibitory concentrations in the NFκB in vitro assay. The most potent inhibitor was maceneolignan J 20 (44). In addition, licarin A (42) and erythro-8.4′-oxyneolign-8′-ene (43) were also more potent NFκB inhibitors than the positive control rocaglamide (IC50=75 nM).

While the natural products (41-44) were considerably more active than the derivatives (45-48), the natural compounds and derivatives were both evaluated for antidiabetic potential in the larval diabetic zebrafish assay. The in vivo assay facilitated analysis of NFκB, PPAR-γ, and several metabolically-relevant enzymes and proteins in a live, metabolizing organism.

### Bioactivity of Mace Isolates and Derivatives in Diabetic Zebrafish

In a preliminary experiment, mace isolates 41, 42, and 43 were evaluated by Western blot in the larval diabetic zebrafish assay (FIG. 4) to determine their effect on expression of glycated hemoglobin levels and the molecular targets PPAR-γ and NFκB (FIG. 5) Maceneolignan J (44) was not tested in the zebrafish assay due to the presence of a contaminating isomer.

According to the HbA_{1C} band densities (FIG. 5), diabetic zebrafish treated with all of the three mace isolates expressed lower glycated hemoglobin levels than non-diabetic zebrafish, indicating that the natural product compounds returned the diabetic fish to normoglycemia. Of particular note, licarin B (41)-treated diabetic larvae exhibited HbA_{1C} levels even lower than those treated with the thiazolidinedione rosiglitazone, indicating a significant hyperglycemic effect, or reduction in blood-glucose levels. Since 41 did not perform as well as 42 in the in vitro PPAR-γ assay (Table 14) or as well as 42 and 43 in the in vitro NFκB assay (Table 13), it was surprising to see such a dramatic effect. However, this preliminary experiment was not performed in replicate (aside from the pooled group of 10 fish per test sample), so without repeating the experiment, it is possible that the encouraging results may have been due to a more direct effect on blood sugar levels than through NFκB, the activity was an anomaly, or it is an instance of a complex, metabolizing organism responding differently than an in vitro system.

While licarin A (42) exhibited excellent antidiabetic potential in vitro (30.6 nM NFκB IC50 and 81% as effective as rosiglitazone in the PPAR-γ assay), it was the least effective of the three natural products at reducing HbA_{1C} expression in diabetic zebrafish. However, it was consistent with the PPAR-γ ELISA results in that it did not perform as well as rosiglitazone but exhibited lower HbA_{1C} expression (corresponding to a lower blood glucose level) than untreated, diabetic zebrafish. As the parent compound of four derivatives that were also evaluated in the diabetic zebrafish assay, 42 was studied more extensively by zebrafish immunoblot, as discussed in the next section, to increase the statistical significance of the preliminary results and as a potential lead for antidiabetic drug development.

The PPAR-γ band density results (FIG. 4) revealed that diabetic zebrafish treated with either 42 or 43 induced greater PPAR-γ expression that 41. Compound 43 exceeded the expectations indicated from the PPAR-γ in vitro results (Table 14), but the in vitro assay is a measure of specific binding, whereas the zebrafish immunoblot reveals the gene response after treatment with the sample. Therefore, both 42 and 43 appeared to significantly affect PPAR-γ expression in comparison to untreated diabetic fish (B), but neither compound superseded the FDA-approved antidiabetic treatment group (C). Since 41 did not upregulate PPAR-γ expression while it managed to significantly reduce HbA_{1C} levels (FIG. 4), licarin B (41) must exert its hypoglycemic effect through a different pathway.

The band density results for NFκB expression indicated that each mace isolate 41-43 significantly inhibited NFκB expression. Furthermore, the levels of inhibition were consistent with the order of the in vitro results (42 IC50 30.6 nM<43, 64.4 nM<41, 474.8 nM). In summary of the results obtained by preliminary diabetic zebrafish Western blot, for compounds 41-43 isolated from the aril of M. fragrans, licarin B (41) had an exceptional hypoglycemic effect that did not appear to act through PPAR-γ, 42 and 43 exerted increased PPAR-γ expression in comparison to untreated diabetic zebrafish, and all three compounds 41-43 inhibited NFκB expression. Thus, 41 should be investigated to determine the source of its hypoglycemic activity and 41-43 should be further evaluated as potential anti-inflammatory agents.

### HbA_{1C} Bioactivity of Licarin A (42) and its Derivatives (45-48) in Diabetic Zebrafish

Through Western blot analysis, HbA_{1C} band densities (FIG. 7) were analyzed for larval diabetic zebrafish treated licarin A (42) and four of its derivatives (45-48). Diabetic zebrafish treated with the positive control, rosiglitazone, exhibited the lowest expression of HbA_{1C}, demonstrating the hypoglycemic effect that contributed toward its marketability as an FDA-approved antidiabetic drug. However, the non-diabetic control group exhibited higher glycated hemoglobin expression than anticipated, and with a fairly large deviation between blot duplicates, the healthy, untreated (A) group of fish may require reevaluation with a greater number of replicates. It is important to note than in analogous immunoblot experiments group A had sufficiently low HbA_{1C} expression levels, appropriately corresponding to decreased blood-glucose levels.

Licarin A and its derivatives (42 and 45-48) exhibited varying degrees of hypoglycemic effects, with 46, followed closely by 42, as the most effective test samples for decreasing HbA_{1C}. Overall, 42 was the best (of eight neolignans, 41-48) agonist in the PPAR-Y ELISA and the second most effective NFκB inhibitor (IC50=30.6 nM). Furthermore, the HbA_{1C} results for 42 were consistent with those in the preliminary investigation of natural products 41-43 in diabetic zebrafish, as well as the PPAR-γ in vitro assay, where 42 demonstrated potential antidiabetic effects that were not as strong as rosiglitazone but an improvement from the negative control groups.

Derivatives 45, 47, and 48 did not reduce HbA_{1C} expression in comparison to the untreated, diabetic group of zebrafish, indicating little to no hypoglycemic activity. Solubility of these derivatives in the fish water may be a point of consideration.

### PPAR-γ Bioactivity of Licarin A (42) and its Derivatives (45-48) in Diabetic Zebrafish

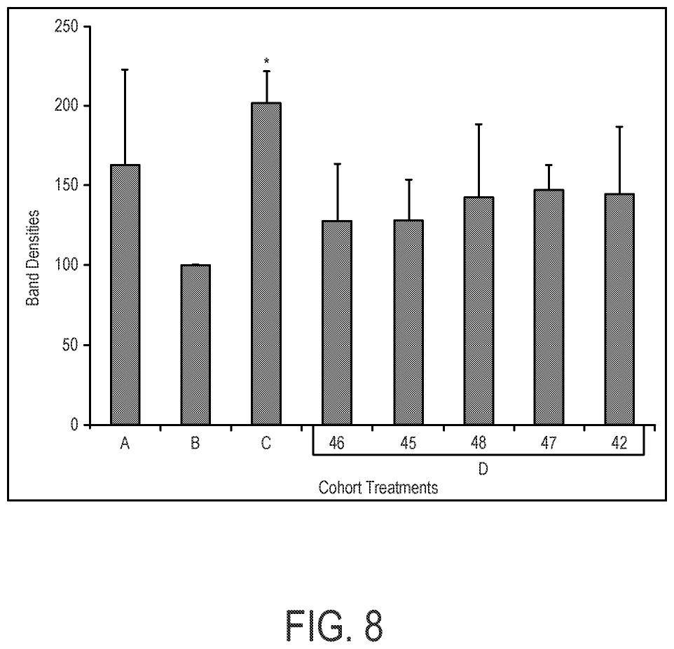

As part of the investigation for potential antidiabetic leads from the licarin A derivative immunoblot, expression of PPAR-γ was evaluated (FIG. 8). As expected, the non-diabetic zebrafish (A) and the rosiglitazone-treated diabetic (C) groups exhibited high PPAR-γ expression, indicative of healthy glucose metabolism and PPAR-γ agonism, respectively. Each of the compounds tested, 42 and 45-48 demonstrated greater PPAR-Y expression than the diabetic, untreated group (B), but none of them surpassed the rosiglitazone (C) or the healthy, untreated (A) control groups. While 42 and 45 were expected to show some PPAR-γ agonism based on the ELISA results (Table 14 and Table 19), 47 performed surprisingly well (and consistently) in the duplicate blots for PPAR-γ. Since the PPAR-γ in vitro assay was only performed as an initial screening and it evaluates specific binding rather than gene response, it is possible that 47 may be an example of a molecule performing differently in vivo.

When comparing the diabetic zebrafish Western blot data of licarin A (42) between the preliminary experiment and the duplicate blot of derivatives, 42 was reliably intermediate between the rosiglitazone-treated and untreated diabetic groups. The only variation in PPAR-γ expression among treatment groups tested in both the preliminary natural product and duplicate derivative blots was that the healthy, untreated group (A) and the rosiglitazone-treated diabetic group (C) fluctuated as having the higher level. However, both responses were acceptable with group A demonstrating normal glucose metabolism or group C showing an excessive gene response while undergoing equilibration back to normoglycemia.

### NFκB Bioactivity of Licarin A (42) and its Derivatives (45-48) in Diabetic Zebrafish

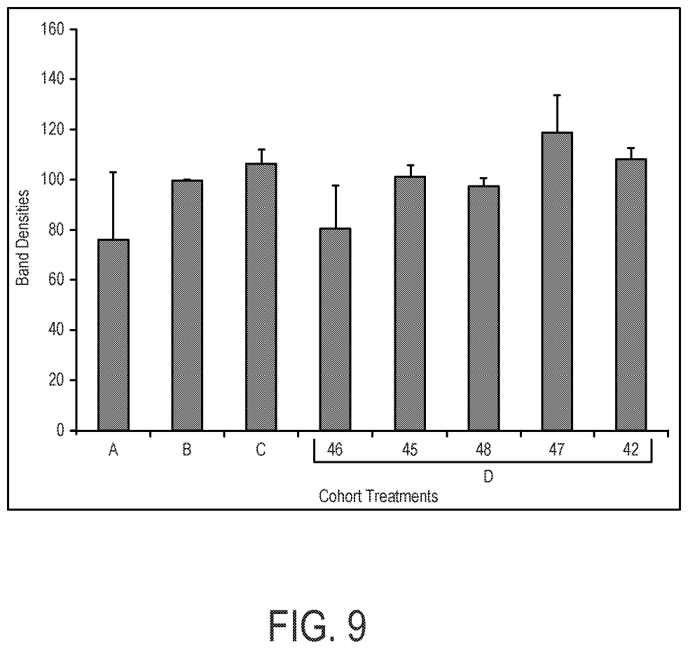

Inhibition of the nuclear transcription factor, NFκB, was investigated in neolignan-treated diabetic zebrafish through Western blot analysis (FIG. 9). The non-diabetic control group (A) exhibited the lowest NFκB expression, consistent with healthy fish and no inflammation-related disease. Derivatives 46 and 48 expressed lower levels of NFκB, but only 46 was statistically significant between the disease negative (A) and positive (B) control groups. Unfortunately, the positive treatment group (C) did not exhibit decreased NFκB expression, but rosiglitazone is not well-known for its NFκB inhibitory effects as it acts through a PPAR-γ agonism mechanism instead.

According to the NFκB results, only 42 performed well in the preliminary natural product zebrafish blot. While 42 should be retested due to the opposing NFκB diabetic zebrafish Western blot results, 47 deserves further investigation in the zebrafish assay to verify the results and to explore the possibility that it could be exerting an anti-inflammatory mechanism that suits chronic disease-related inflammation.

### PEPCK Bioactivity of Licarin A (42) and its Derivatives (45-48) in Diabetic Zebrafish

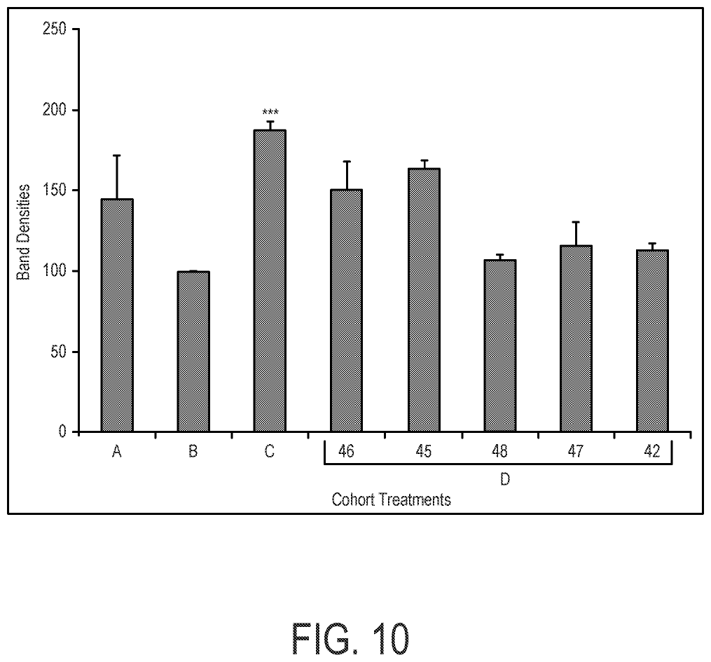

With an ideal PEPCK expression level being equal to or slightly elevated in comparison to non-diabetic zebrafish (A), diabetic larvae treated with rosiglitazone (C), 45 or 46 indicated a return to normoglycemia (FIG. 10). While 42, 47, and 48 retained low PEPCK expression consistent with a hold on gluconeogenesis, none of the compounds caused concern for hypoglycemia. Moreover, none of the test compounds were exhibited better PEPCK levels than the positive control rosiglitazone (C), but they were all at least marginally better than the untreated diabetic group (B). Therefore, depending on their immunoblot results for the other targets, they may still be studied as possible antidiabetic or anti-inflammatory candidates.

### Glut-4 Bioactivity of Licarin A (42) and its Derivatives (45-48) in Diabetic Zebrafish

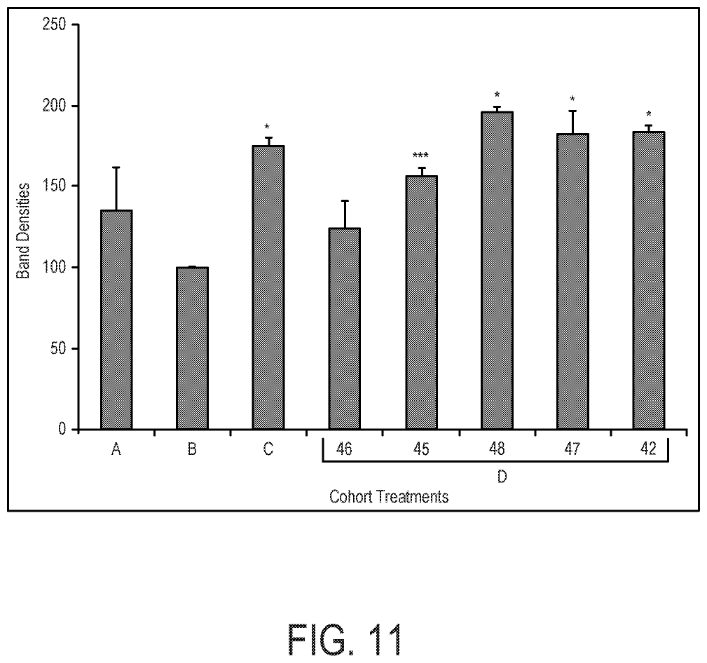

Since thiazolidinediones are known to increase expression of glucose transporters, the GLUT-4 expression of rosiglitazone-treated zebrafish in FIG. 11 was as expected and demonstrates an increased capacity for glucose uptake. Impressively, all of the neolignans 42 and 45-48 exhibited greater GLUT-4 expression than the untreated, diabetic group (B) of zebrafish. In particular, compounds 42, 47, and 48 showed significantly increased capacities for glucose uptake. These results may not directly indicate potential antidiabetic leads, but they allow for that opportunity if there are other indicators of the compounds exerting hypoglycemic effects.

In summary of the derivative zebrafish blot, compounds 42 and 46 exhibited minor hypoglycemic activities. Licarin A (42) and each of its derivatives (45-48) appeared to have intermediate PPAR-γ activities. Compound 46 inhibited NFκB expression while 42 and 47 require further NFκB analysis. Derivatives 45 and 46 appeared to cause type 2 diabetic zebrafish to return to normoglycemia by comparison of PEPCK expression levels, and all of the compounds tested (particularly 42, 47, and 48) showed an increased expression of GLUT-4, indicating a greater capacity for glucose uptake. Therefore, derivative 46 was the best of those tested for possible antidiabetic activity, and compounds 42 and 47 require further evaluation as possible antidiabetic or anti-inflammatory leads.

### Conclusions

Myristica fragrans seeds and arils have been used extensively in culinary settings, as well as in traditional medicine for various disease conditions, including digestive disorders, rheumatism, and several other inflammation-related conditions. Type 2 diabetes, as an inflammatory disease highly correlated to diet and nutrition, was identified as a disease state that necessitated drug discovery and development for products with fewer side effects. Appropriately, the edible spice was assigned as the source of material for antidiabetic drug discovery and development efforts, and M. fragrans arils were obtained and screened for antidiabetic activity.

The ethyl acetate partition of a methanolic macerate produced from M. fragrans arils was fractionated, and D12101-F005 was screened in vitro for anti-inflammatory effects acting through the molecular target NFκB, when it was identified as active by surpassing the assay positive control. The same fraction also exerted PPAR-γ activity in the in vitro assay that was nearly equal to the FDA-approved antidiabetic PPAR-γ agonist, rosiglitazone. Two subfractions (D12101-F020 and D12101-F027) formed precipitates, which were isolated and identified as known compounds licarin A (42) and licarin B (41). Both compounds were evaluated in the NFκB in vitro assay, where they exhibited half maximal inhibitory concentrations (42 IC50=30.6 nM; 41 IC50=474.8 nM) better than the positive control rocaglamide (IC50=75 nM). Mace isolates 41 and 42 were also screened for PPAR-γ activity, where 42 warranted further research by achieving 81% PPAR-γ activation in comparison to rosiglitazone. The slightly lower PPAR-γ activity of 42 was hypothesized to indicate a partial agonist, where the weaker ligand-receptor interactions may limit the number and severity of side effects.

Due to the exceptional bioactivity of 41 and 42, neighboring fractions were combined and sub-fractionated until two additional compounds were isolated by reversed-phase HPLC and identified as known compounds, erythro-8.4′-oxyneolign-8′-ene (43) and maceneolignan J (44). Thus, bioactivity-guided fractionation of this sample was conducted, and led to the isolation of four known compounds, including two 8,3′-type neolignans (41 and 42) and two 8-O-4′-type neolignans (43 and 44).

The two additional mace isolates (43 and 44) were also analyzed for antidiabetic potential in vitro, exerting only moderate PPAR-γ activity but significant inhibition of NFκB (43 IC50=64.4 nM; 44 IC50=0.1 nM). The NFκB and PPAR-γ activity observed for these isolated compounds, either alone or acting in synergy with others, may explain the biological activity observed for the crude extract and ethyl acetate partition, and may also help to resolve the principal bioactive molecules that justify the use of M. fragrans in traditional medicines.

The biological activities observed for 41-44 suggested that these four compounds may have potential as antidiabetic agents or lead molecules for medicinal chemistry optimization. The compound most active in the entire in vitro panel of assays, which was also the most abundant isolate (42), was therefore derivatized through etherification and esterification reactions to yield four new neolignans, 45-48. The new compounds were herein referred to as isopropoxy-licarin A (45), isopropoxy-licarin A carbaldehyde (46), licarin A ethyl carbonate (47), and licarin A formyl ethyl carbonate (48), based on their structural relationships to the parent compound, licarin A (42).

Derivatives 45-48 were further investigated, along with three of the mace isolates (41-43), in the newly developed larval diabetic zebrafish assay to explore the structure-activity relationships, lead development, and possible mechanisms of the compounds. The study also provided an opportunity to validate the new diabetic zebrafish assay as a way to evaluate natural product compounds in a semi-high-throughput manner, especially for those isolated in small yields but still deserving of in vivo investigation.

After developing the larval diabetic zebrafish assay, the new in vivo model was simultaneously validated and used to evaluate the M. fragrans compounds and associated derivatives. Expression levels of metabolic enzymes and molecular targets for the assay controls met and followed expectations according to their biochemical relationships. In the preliminary Western blot experiment with the diabetic zebrafish, contradictions between results of each compound at different targets revealed the necessity of incorporating increased replicates. The moderate activity of 42 according to HbA_{1C} expression, PPAR-γ activation, and inhibition of the NFκB-mediated inflammatory response perpetuated the hypothesis of the compound acting as a partial agonist and whose hypoglycemic mechanism was related to both PPAR-γ and NFκB. Additionally, the diabetic zebrafish Western blot performed in duplicate to evaluate the potential antidiabetic effects of the licarin A derivatives (45-48) yielded two interesting discoveries. Isopropoxy-licarin A carbaldehyde (46) was identified by its significant reduction of HbA_{1C} expression in 46-treated diabetic zebrafish despite exhibiting no significant PPAR-γ activity in vitro. The hypoglycemic activity of 46 is therefore interesting for further studies particularly focused on its mechanism that avoided the interconnected molecular targets NFκB and PPAR-γ. Licarin A ethyl carbonate (47) also gained recognition when this compound exhibited both NFκB and PPAR-γ activity in vivo after displaying only weak NFκB inhibitory activity in HeLa cells. Despite affecting the two in vivo targets in a manner that would indicate a good antidiabetic drug lead, according to the HbA_{1C} and PEPCK expression levels, 47 was not at all effective at returning the diabetic organism to normoglycemia. Since it does not improve glucose metabolism but affects the targets, the mechanism of 47 may lend itself more toward the anti-inflammatory role rather than antidiabetic. In addition, variation in activity between the in vitro and in vivo observations for the licarin A derivatives (45-48) may be a result of reduced solubility in water following their aliphatic modifications.

Licarin B (41) had excellent hypoglycemic effect but did not work through PPAR-γ, and 42 and 46 exhibited slight hypoglycemic effects. Compounds 42-48 increased PPAR-Y expression to levels intermediating diabetic zebrafish treated with vehicle (B) and the FDA-approved antidiabetic drug, rosiglitazone (C). Isolates 41-43, as well as derivative 46 inhibited NFκB expression and should be evaluated as possible anti-inflammatory agents. Additionally, further studies of licarin B (41) are necessary to determine its hypoglycemic activity and may or may not lead to the discovery of a novel antidiabetic pathway.

The new diabetic zebrafish assay, which requires similar amounts of material to in vitro assays, highlighted two new compounds for further studies that would have been overlooked if dependent only on in vitro results. The new model also allowed mechanistic probing of several enzymes, receptors, and macromolecules through a singular experiment.

### Preliminary Docking Analysis of Derivatives Compared to Parent Compound

| | | Estimated free | | |
|---|---|---|---|---|
| | | energy of | Estimated | |
| | | binding | inhibition | Binding Pocket Amino |
| Compound | Protein | (kcal/mol) | constant (nM) | Acids |
| LicA-R2P2.1 | PPAR-G | −8.77 | 375.92 | Leu22, Ala292, Leu340, |
| | | | | Ser342 |
| LicA-R2P2.2 | PPAR-G | −9.35 | 141.06 | Ile341, Glu343, Ser332, |
| | | | | Leu348 |
| LicA-R3P1 | PPAR-G | −8.3 | 819.13 | Glu295, Leu228, Glu284, |
| | | | | Arg288 |
| LicA-R3P3 | PPAR-G | −8.82 | 345.38 | Leu340, Arg288, Glu343, |
| | | | | Ala292 |
| Licarin | PPAR-G | −7.15 | 5.75 | Leu330, Ile341, Leu228, |
| | | | | Met329 |
| LicA-R2P2.1 | NFκB- | −6.76 | 11050 | Ser281, Glu282, |
| | p65 | | | Glu286Lys195 |
| LicA-R2P2.2 | NFκB- | −6.73 | 11680 | Ile224, Glu237, Glu222, |
| | p65 | | | Lys221 |
| LicA-R3P1 | NFκB- | −6.88 | 9060 | Ser281, Glu282, Met284, |
| | p65 | | | Lys196 |
| LicA-R3P3 | NFκB- | −0.25 | 9060 | Ser281, Glu282, Met284, |
| | p65 | | | Lys196 |
| Licarin | NFκB- | −6.82 | 10060 | Glu222, Lys221, Ser240, |
| | p65 | | | Gln241 |

These derivatives were also screened in silico against other target(s) (PARP-5/PEPCK) and data collected show their potential against these targets.

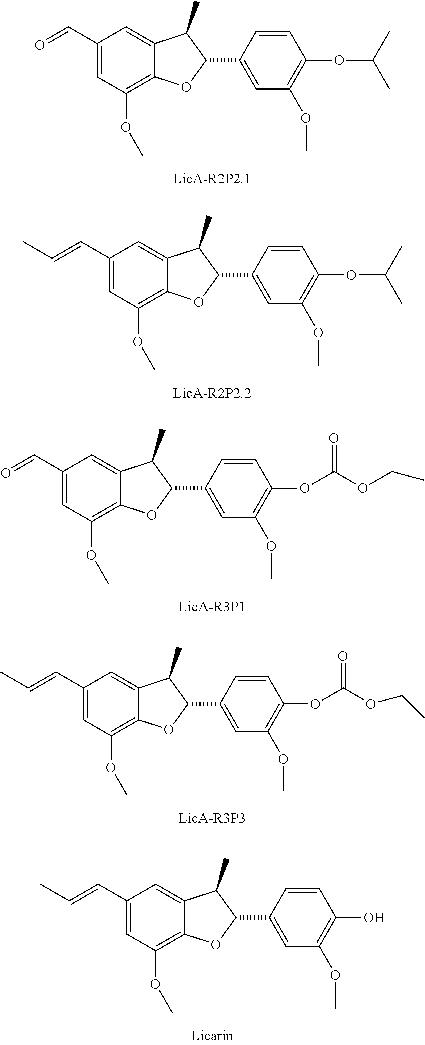

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A compound of Formula I, Formula II, Formula III, or Formula IV: 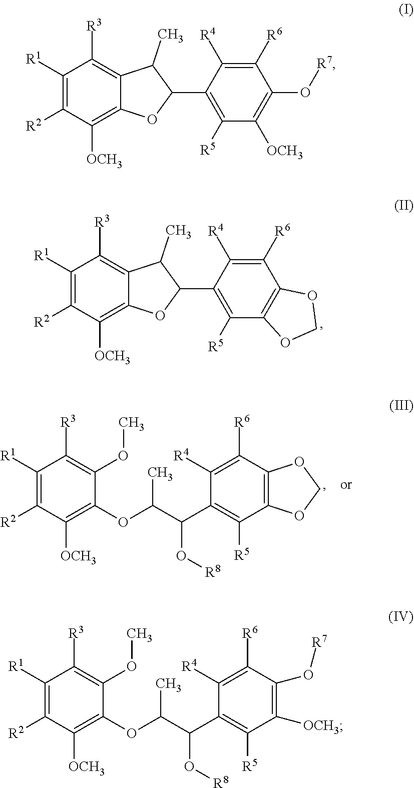
or a pharmaceutically acceptable salt, prodrug, or derivative thereof;
wherein:
R¹ is selected from hydrogen, alkyl, alkenyl, alkynyl, aryl, heteroaryl, cycloalkyl, cycloalkenyl, aldehyde, carboxylic acid, ester, ketone, amino, hydroxy, alkoxy, nitro, and thiol, each of which may be optionally substituted with one or more substituents as allowed by valence independently selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, and thiol;
R², R³, R⁴, R⁵, and R⁶ are each independently selected from hydrogen, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, and thiol, each of which R², R³, R⁴, R⁵, and R⁶ may be optionally substituted with one or more substituents as allowed by valence independently selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol;
R⁷ is selected from hydrogen, alkyl, aryl, heteroaryl, cycloalkyl, C(O)R⁹, P(O)(R⁹)₂, and S(O)₁₋₂R⁹, each of which may be optionally substituted with one or more substituents as allowed by valence independently selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol;
R⁸ is selected from hydrogen, alkyl, aryl, heteroaryl, cycloalkyl, C(O)R⁹, P(O)(R⁹)₂, and and S(O)₁₋₂R⁹, each of which may be optionally substituted with one or more substituents as allowed by valence independently selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol; and
R⁹ is selected independently at each occurrence from hydrogen, alkyl, halogenated alkyl, alkoxy, alkenyl, alkynyl, aryl, heteroaryl, and amino, each of which R⁹ may be optionally substituted with one or more substituents as allowed by valence independently selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol;
with the proviso that the compound of Formula I cannot be: 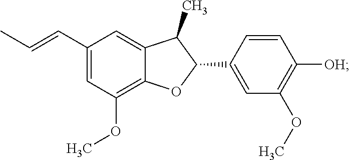 [SMILES: C/C=C/c1cc(OC)c2c(c1)[C@@H](C)[C@H](c1ccc(O)c(OC)c1)O2]
the compound of Formula II cannot be: 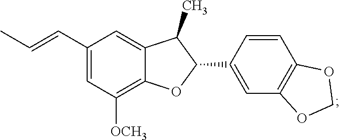 [SMILES: C/C=C/c1cc(OC)c2c(c1)[C@@H](C)[C@H](c1ccc3c(c1)OCO3)O2]
and
the compound of formula III cannot be:

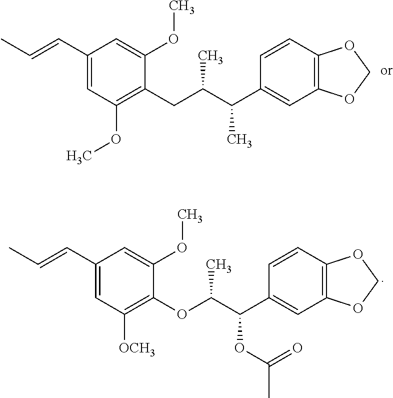 [SMILES: C/C=C/c1cc(OC)c(C[C@H](C)[C@@H](C)c2ccc3c(c2)OCO3)c(OC)c1 ; C/C=C/c1cc(OC)c(O[C@H](C)[C@@H](OC(C)=O)c2ccc3c(c2)OCO3)c(OC)c1]

2. The compound of claim 1, wherein the compound is of Formula Ia, Formula Ib, Formula IIa, Formula IIb, Formula IIIa, Formula IIIb, Formula IVa, or Formula IVb: 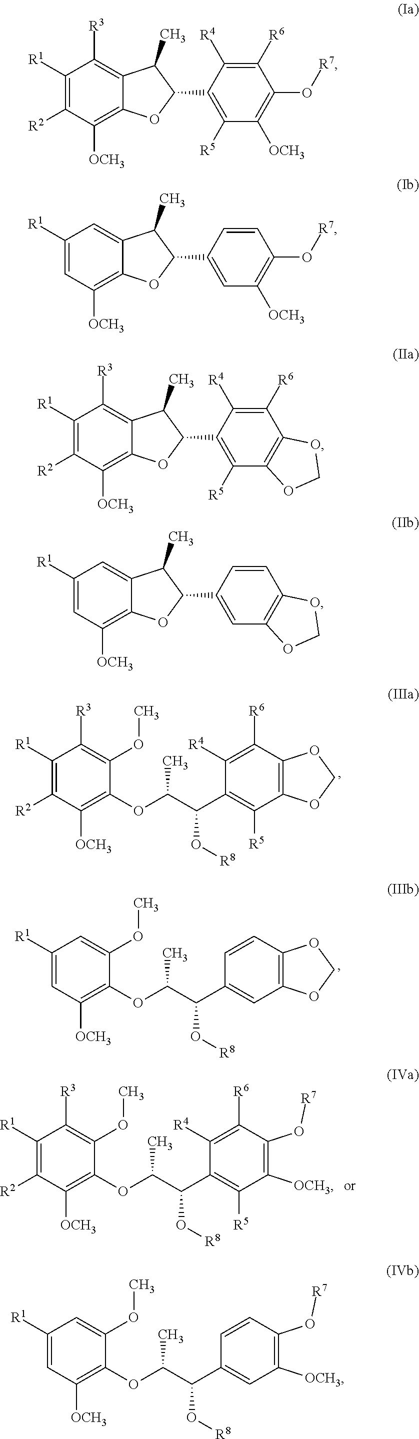
or a pharmaceutically acceptable salt, prodrug, or derivative thereof.

3-9. (canceled)

10. The compound of claim 1, wherein R², R³, R⁴, R⁵, and R⁶ are each independently selected from hydrogen, halogen, or C₁-C₆ alkyl.

11. The compound of claim 10, wherein R² is selected from hydrogen, fluoro, chloro, bromo, iodo, and methyl, or
wherein R³ is selected from hydrogen, fluoro, chloro, bromo, iodo, and methyl, or
wherein R⁴ is selected from hydrogen, fluoro, chloro, bromo, iodo, and methyl, or
wherein R⁵ is selected from hydrogen, fluoro, chloro, bromo, iodo, and methyl, or
wherein R⁶ is selected from hydrogen, fluoro, chloro, bromo, iodo, and methyl.

12-15. (canceled)

16. The compound of claim 1, wherein R⁷ is selected from C₁-C₆ alkyl and C(O)R⁹.

17. The compound of claim 16, wherein R⁷ is selected from methyl, ethyl, n-propyl, or isopropyl, or
wherein R⁷ is C(O)(R⁹), wherein R⁹ is methyl, ethyl, n-propyl, isopropyl, phenyl, methoxy, ethoxy, n-propoxy, and isopropoxy.

18-19. (canceled)

20. The compound of claim 1, wherein R⁸ is selected from C₁-C₆ alkyl and C(O)R⁹.

21. The compound of claim 20, wherein R⁸ is selected from methyl, ethyl, n-propyl, or isopropyl, or
wherein R⁸ is C(O)(R⁹), wherein R⁹ is methyl, ethyl, n-propyl, isopropyl, phenyl, methoxy, ethoxy, n-propoxy, and isopropoxy.

22-23. (canceled)

24. The compound of claim 1, wherein R¹ is selected from C₂-C₆ alkenyl and C(O) H.

25. The compound of claim 1, wherein the compound is selected from: 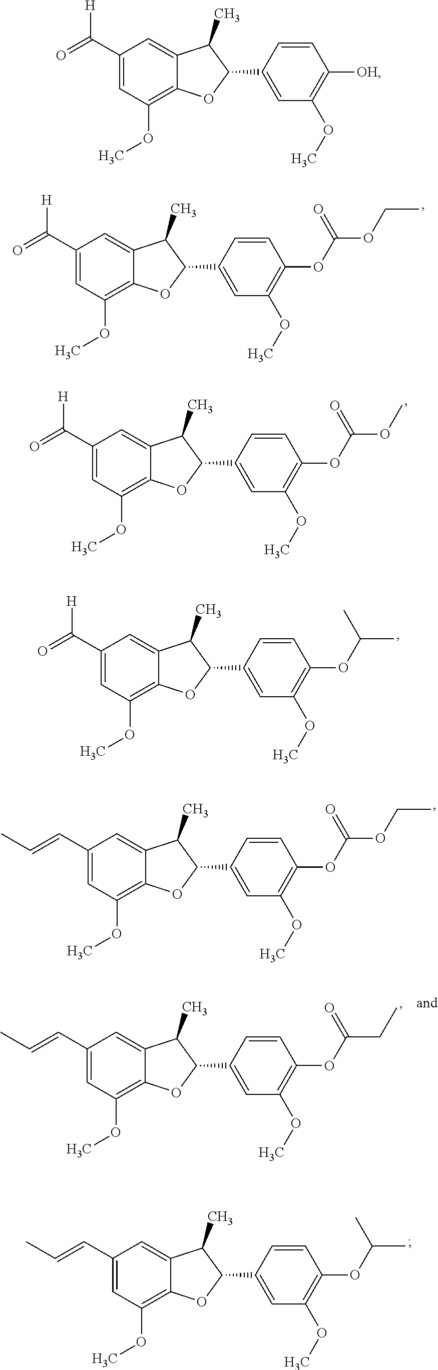
or a pharmaceutically acceptable salt, prodrug, or derivative thereof.

26. A pharmaceutical composition comprising a compound of claim 1, or a pharmaceutically acceptable salt, prodrug, or derivative thereof, in a pharmaceutically acceptable carrier.

27. A method of treating a metabolic disorder in a subject in need thereof comprising administering a therapeutically effective amount of a compound of claim 1 to the subject, or a pharmaceutically acceptable salt, prodrug, or derivative thereof.

28. The method of claim 27, wherein the metabolic disorder is diabetes, obesity, metabolic syndrome, diabetic dyslipidemia, or hypertriglyceridemia.

29-32. (canceled)

33. A method of treating hyperglycemia in a subject in need thereof comprising administering a therapeutically effective amount of a compound of claim 1 to the subject, or a pharmaceutically acceptable salt, prodrug, or derivative thereof.

34. A method of treating inflammation in a subject in need thereof comprising a therapeutically effective amount of a compound of claim 1 to the subject, or a pharmaceutically acceptable salt, prodrug, or derivative thereof.

35. The method of claim 34, wherein the inflammation is acute inflammation or chronic inflammation.

36. (canceled)

37. The method of claim 34, wherein the inflammation is associated with an inflammatory disorder, arthritis, a gastrointestinal condition, or a systemic inflammatory disorder.

38-46. (canceled)

47. A method of treating a metabolic disorder or inflammation in a subject in need thereof comprising administering a therapeutically effective amount of a compound selected from a compound of Formula A, Formula B, Formula C, or Formula D: 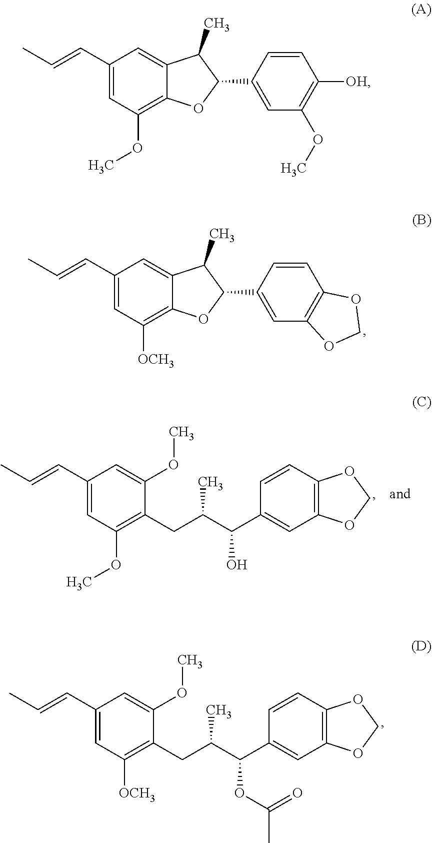 [SMILES: C/C=C/c1cc(OC)c2c(c1)[C@@H](C)[C@H](c1ccc(O)c(OC)c1)O2 ; C/C=C/c1cc(OC)c2c(c1)[C@@H](C)[C@H](c1ccc3c(c1)OCO3)O2 ; C/C=C/c1cc(OC)c(C[C@H](C)[C@@H](O)c2ccc3c(c2)OCO3)c(OC)c1 ; C/C=C/c1cc(OC)c(C[C@H](C)[C@@H](OC(C)=O)c2ccc3c(c2)OCO3)c(OC)c1]
or a pharmaceutically acceptable salt, prodrug, or derivative thereof.

48. The method of claim 47, wherein the metabolic disorder is diabetes, obesity, metabolic syndrome, diabetic dyslipidemia, or hypertriglyceridemia.

49-53. (canceled)

54. The method of claim 47, wherein the inflammation is acute inflammation or chronic inflammation.

55. (canceled)

56. The method of claim 47, wherein the inflammation is associated with an inflammatory disorder, arthritis, a gastrointestinal condition, or a systemic inflammatory disorder.

57-65. (canceled)
